(19) United States
(12) Patent Application Publication
(10) Pub. No.: US 2020/0086324 A1
(43) Pub. Date: Mar. 19, 2020

(54) DEVICES AND METHODS FOR NUCLEIC ACID EXTRACTION

(71) Applicant: Visby Medical, Inc., San Jose, CA (US)

(72) Inventors: David D. Swenson, Santa Clara, CA (US); Adam De La Zerda, Palo Alto, CA (US); Gregory C. Loney, Los Altos, CA (US); Valeria Revilla, East Palo Alto, CA (US); Victor Briones, Gilroy, CA (US); Boris Andreyev, Foster City, CA (US); Adrienne C. Lam, Fremont, CA (US); Brian Ciopyk, Pleasanton, CA (US); Helen Huang, San Pablo, CA (US); Keith Moravick, Mountain View, CA (US); Colin Kelly, San Francisco, CA (US); Jesus Ching, Saratoga, CA (US); Jennifer Albrecht, Sunnyvale, CA (US); Ryan Cena, San Jose, CA (US); Edward Biba, Santa Clara, CA (US); Jonathan Hong, San Jose, CA (US)

(21) Appl. No.: 16/234,453

(22) Filed: Dec. 27, 2018

Related U.S. Application Data
(63) Continuation of application No. PCT/US2017/040112, filed on Jun. 29, 2017.
(60) Provisional application No. 62/357,306, filed on Jun. 30, 2016.

(51) Int. Cl. B01L 7/00; B01L 3/00; C12Q 1/6806; C12Q 1/6844

(52) U.S. Cl. CPC B01L 7/52; B01L 3/502715; C12Q 1/6806; B01L 2200/10; B01L 3/502769; C12Q 1/6844; B01L 2200/027; B01L 3/502761

(57) ABSTRACT

Disclosed herein are methods and devices for preparing a sample of nucleic acid molecules from a biological sample. The methods and devices may perform similarly to or better than standard sample preparation methods. The nucleic acid molecules prepared using the methods and devices provided herein may be utilized for downstream applications, including polymerase chain reaction (PCR).

### CROSS-REFERENCE

This application claims the benefit of U.S. Provisional Application No. 62/357,306, filed Jun. 30, 2016, which application is incorporated herein by reference.

### BACKGROUND OF THE INVENTION

The embodiments described herein relate to methods and devices for molecular diagnostic testing. More particularly, the embodiments described herein relate to disposable, self-contained devices and methods for molecular diagnostic testing. Particular embodiments described herein relate to disposable, self-contained devices and methods for purifying, reverse transcribing and detecting nucleic acids.

There are over one billion infections in the U.S. each year, many of which are treated incorrectly due to inaccurate or delayed diagnostic results. Many known point of care (POC) tests have poor sensitivity (30-70%), while the more highly sensitive tests, such as those involving the specific detection of nucleic acids or molecular testing associated with a pathogenic target, are only available in laboratories. Thus, approximately ninety percent of the current molecular diagnostics testing is practiced in centralized laboratories. Known devices and methods for conducting laboratory-based molecular diagnostics testing, however, require trained personnel, regulated infrastructure, and expensive, high throughput instrumentation. Known laboratory instrumentation is often purchased as a capital investment along with a regular supply of consumable tests or cartridges. Known high throughput laboratory equiμment generally processes many (96 to 384 and more) samples at a time, therefore central lab testing is done in batches. Known methods for processing typically include processing all samples collected during a time period (e.g., a day) in one large run, with a turn-around time of hours to days after the sample is collected. Moreover, such known instrumentation and methods are designed to perform certain operations under the guidance of a skilled technician who adds reagents, oversees processing, and moves sample from step to step. Thus, although known laboratory tests and methods are very accurate, they often take considerable time, and are very expensive.

There are limited testing options available for testing done at the point of care (“POC”), or in other locations outside of a laboratory. Known POC testing options tend to be single analyte tests with low analytical quality. These tests are used alongside clinical algorithms to assist in diagnosis, but are frequently verified by higher quality, laboratory tests for the definitive diagnosis. Thus, neither consumers nor physicians are enabled to achieve a rapid, accurate test result in the time frame required to “test and treat” in one visit. As a result doctors and patients often determine a course of treatment before they know the diagnosis. This has tremendous ramifications: antibiotics are either not prescribed when needed, leading to infections; or antibiotics are prescribed when not needed, leading to new antibiotic-resistant strains in the community. Moreover, known systems and methods often result in diagnosis of severe viral infections, such as H1N1 swine flu, too late, limiting containment efforts. In addition, patients lose much time in unnecessary, repeated doctor visits.

Thus, a need exists for improved devices and methods for molecular diagnostic testing. In particular, a need exists for an affordable, easy-to-use test that will allow healthcare providers and patients at home to diagnose infections accurately and quickly so they can make better healthcare decisions.

### SUMMARY OF THE INVENTION

In one aspect, a molecular diagnostic test device includes a housing, a reverse transcription module, an amplification module and a detection module. The reverse transcriptase module is configured to receive an input sample and includes a heater such that the reverse transcription module can perform a reverse transcriptase polymerase chain reaction (RT-PCR) on the input sample. The amplification module is configured to receive a cDNA sample from the reverse transcription module. The amplification module includes a heater such that the amplification module can perform a polymerase chain reaction (PCR) on the input sample. The detection module is configured to receive an output from the amplification module and a reagent formulated to produce a signal that indicates a presence of a target amplicon within the input sample. The reverse transcription module, amplification module and the detection module are integrated within the housing such that the molecular diagnostic test device is a handheld device.

In some cases, the signal is a non-fluorescent signal. In some cases, the signal is a visible signal characterized by a color associated with the presence of the target amplicon; and the detection module includes a detection surface from which the visible signal is produced, the detection surface visible via a detection opening defined by the housing. In some cases, the signal is a visible signal characterized by a color associated with the presence of the target amplicon, the reagent formulated such that the visible signal remains present for at least about 30 minutes.

In some cases, the molecular diagnostic test device further comprises a power source disposed within the housing and configured to supply power to the amplification module, the power source including a DC battery having a nominal voltage of about 9V, the power source having a capacity of less than about 1200 mAh.

In some cases, the molecular diagnostic test device further comprises a power source disposed within the housing; and a reagent module disposed within the housing, the reagent module including a sealed volume within which the reagent is contained, the reagent module including a reagent actuator configured to convey the reagent into a holding chamber fluidically coupled to the detection module when the reagent actuator is moved from a first position to a second position, the power source being electrically isolated from the amplification module when the reagent actuator is in the first position, the power source being electrically coupled to at least one of a processor or the amplification module when the reagent actuator is in the second position. In some cases, the molecular diagnostic test device further comprises a sample input module disposed within the housing, the sample input module including an inlet port, an outlet port, the inlet port configured to receive the input sample; and a sample actuator configured to convey the input sample via the outlet port and through a filter assembly when the sample actuator is moved from a first position to a second position, the sample actuator configured to remain locked in the second position. In some cases, the sample actuator is in a fixed position relative to at least one of the amplification module or the detection module when the sample actuator is in the second position. In some cases, the sample actuator is a non-electronic actuator configured to move irreversibly from the first position to the second position. In some cases, the molecular diagnostic test device is configured for one and only one use and is disposable.

In another aspect an apparatus comprises a housing defining a detection opening; a reverse transcription module disposed within the housing, the reverse transcription module including a flow member and a heater, the flow member defining an reverse transcription flow path having an inlet portion configured to receive a sample, the heater fixedly coupled to the flow member such that the heater and the amplification flow path intersect at multiple locations; an amplification module disposed within the housing, the amplification module including a flow member and a heater, the flow member defining an amplification flow path having an inlet portion configured to receive a sample, the heater fixedly coupled to the flow member such that the heater and the amplification flow path intersect at multiple locations; a reagent module disposed within the housing, the reagent module containing a substrate formulated to catalyze the production of a signal by a signal molecule associated with a target amplicon; and a detection module defining a detection channel in fluid communication with an outlet portion of the amplification flow path and the reagent module, the detection module including a detection surface within the detection channel, the detection surface configured to retain the target amplicon, the detection module disposed within the housing such that the detection surface is visible via the detection opening of the housing. In some cases, the amplification and/or reverse transcription flow path is a serpentine flow path, and the heater is a linear heater irreversibly coupled to the flow member. In some cases, the amplification and/or reverse transcription flow path is a serpentine flow path; the heater is a heater assembly including a first linear heater coupled to a first end portion of the flow member, a second linear heater coupled to a second end portion of the flow member, a third linear heater coupled to a central portion of the flow member, the heater assembly coupled to of a first side the flow member via an adhesive bond.

In some cases, the apparatus further comprises a power source disposed within the housing and configured to supply power to the heater, the power source having a nominal voltage of about 9 VDC and a capacity of less than about 1200 mAh. In some cases, the apparatus further comprises a power module removably coupled to the housing, the power module including a power source having a nominal voltage of about 9 VDC and a capacity of less than about 1200 mAh, the power module including an electronic circuit electrically coupled to the heater when the power module is coupled to the housing.

In some cases, the apparatus further comprises a power source having a nominal voltage of about 9 VDC and a capacity of less than about 1200 mAh; and an isolation member removably coupled to the housing, the power source being electrically isolated from the heater when the isolation member is coupled to the housing, the power source being electrically coupled to the heater when the isolation member is removed from the housing,

the reagent module including a reagent actuator configured to release the substrate into a holding chamber when the reagent actuator is moved from a first position to a second position, the movement of the isolation member being limited when the reagent actuator is in the first position.

In some cases, the apparatus further comprises a power source disposed within the housing, the reagent module including a reagent actuator configured to release the substrate into a holding chamber when the reagent actuator is moved from a first position to a second position, the power source being electrically isolated from the heater when the reagent actuator is in the first position, the power source being electrically coupled to the heater when the reagent actuator is in the second position. In some cases, the apparatus further comprises a controller disposed within the housing, the controller implemented in at least one of a memory or a processor, the controller including a thermal control module configured to produce a thermal control signal to adjust an output of the heater.

In some cases, the signal is a visible signal characterized by a color associated with the presence of the target amplicon; and the detection channel has a width of at least about 4 mm. In some cases, the housing includes a mask portion configured to surround at least a portion of the detection opening, the mask portion configured to enhance visibility of the detection surface through the detection opening.

In some cases, the reagent module includes a reagent formulated to produce the signal; and the signal is a non-fluorescent visible signal characterized by a color associated with the presence of the target amplicon, the reagent formulated such that the visible signal remains present for at least about 30 minutes.

In another aspect an apparatus comprises a housing; a sample preparation module disposed within the housing and configured to receive an input sample, the sample preparation module including a filter assembly; a reverse transcription module disposed within the housing and configured to receive an output from the sample preparation module, the reverse transcription module including a flow member and a heater, the flow member defining an reverse transcription flow path having an inlet portion configured to receive a sample, the heater fixedly coupled to the flow member such that the heater and the amplification flow path intersect at multiple locations; an amplification module disposed within the housing and configured to receive an output from the reverse transcription module, the amplification module including a flow member and a heater, the flow member defining a serpentine flow path, the heater coupled to the flow member, the amplification module configured perform a polymerase chain reaction (PCR) on the output from the sample preparation module; and a detection module disposed within the housing and configured to receive an output from the amplification module, wherein the apparatus is configured for one-time use. In some cases, the detection module is configured to receive a reagent formulated to produce a colorimetric signal that indicates a presence of a target organism in the input sample. In some cases, the apparatus further comprises a sample actuator configured to produce a force to convey the input sample through the filter assembly when the sample actuator is moved from a first position to a second position, the sample actuator configured to remain locked in the second position, the sample actuator including a locking shoulder configured to matingly engage a portion of the housing to maintain the sample actuator in the second position. In some cases, the sample preparation module is fixedly coupled within the housing. In some cases, the detection module is fixedly coupled within the housing and includes a detection surface from which a colorimetric signal that indicates a presence of a target organism in the input sample is produced, the detection surface visible via a detection opening defined by the housing.

In some cases, the apparatus further comprises a fluid transfer module disposed within the housing, the fluid transfer module defining an internal volume within which the output of the sample preparation module flows when the fluid transfer module is actuated, the fluid transfer module configured to convey the output of the sample preparation module from the internal volume to the amplification module, the fluid transfer module being fixedly and fluidically coupled to the sample preparation module. In some cases, the fluid transfer module includes a plunger movably disposed within the internal volume such that movement of the plunger conveys the output of the sample preparation module from the internal volume to the amplification module. In some cases, the apparatus further comprises a power source disposed within the housing and configured to supply power to the amplification module, the power source having a capacity of less than about 1200 mAh. In some cases, the sample preparation module includes a wash container containing a gas wash and a liquid wash, the sample preparation assembly configured to convey the gas wash and the liquid wash through the filter assembly in series, further comprising: a wash actuator configured to produce a force to convey the gas wash through the filter assembly at a first time and the liquid wash through the filter assembly at a second time after the first time when the wash actuator is moved from a first position to a second position.

In some cases, the heating element can heat a liquid in the mixing chamber to a temperature between 20 C and 100 C. In some cases, the heating element can heat a liquid in the mixing chamber to a temperature between 20 C and 50 C. In some cases, the heating element can heat a liquid in the mixing chamber to a temperature between 85 C and 95 C. In some cases, the heating element can hold a liquid in the mixing chamber at a constant temperature between 20 C and 50 C. In some cases, the heating element can hold a liquid in the mixing chamber at a constant temperature between 85 C and 95 C. In some cases, the heating element can hold a liquid in the mixing chamber at a constant temperature for a time between 0.1 to 24 hours. In some cases, the heating element can hold a liquid in the mixing chamber at a constant temperature for a time between 0.1 to 1 hour. In some cases, the heating element can hold a liquid in the mixing chamber at a constant temperature for a time between 1 second and 30 minutes. In some cases, the heating element can hold a liquid in the mixing chamber at a constant temperature for a time between 1 second and 10 minutes. In some cases, the reverse transcription chamber of step (b) further comprises a mixing chamber and a serpentine channel. In some cases, the mixing chamber can hold a volume between 10 ul and 10 ml·s In some cases, the mixing chamber can hold a volume between 10 ul and 1 ml. In some cases, the mixing chamber can hold a volume of 300 ul. In some cases, the serpentine channel is designed to have a cross-section with an aspect ratio (channel height to width) to maximize the area in contact with heater allowing efficient heat coupling to the fluid. In some cases, the device is designed to perform and analyze multiplexed PCRs. In some cases, the reverse transcription module further comprises a lyophilized pellet comprising reverse transcriptase enzyme and reagents. In some cases, the reverse transcription module contains a reagent chamber containing reverse transcriptase enzyme and reagents required for a reverse transcriptase polymerase chain reaction. In some cases, the reverse transcriptase enzyme and reagents are present as a lyophilized pellet. In some cases, the reverse transcriptase enzyme and reagents are present with the DNA polymerase enzyme and PCR reagents.

In another aspect, a method for DNA preparation comprises obtaining a biological sample comprising one or more biological entities comprising RNA; capturing said one or more biological entities on a filter; eluting said one or more biological entities from said filter; and lysing said one or more biological entities, incubating the lysed biological entities with a reverse transcriptase enzyme and sufficient reagents to perform a reverse transcription reaction, thereby preparing a plurality of DNA molecules therefrom, wherein said method prepares said DNA molecules from said one or more biological entities within 10 minutes or less at a quality sufficient to successfully perform a polymerase chain reaction (PCR).

In some cases, the method further comprises that the filter consists of two filter membranes, a first filter membrane and a second filter membrane with a smaller pore size than the first filter membrane.

In some cases, the method further comprises a wash step, whereby once the biological entities are captured on the filter the filter and biological entities are washed with an air wash.

In another aspect, a method for DNA preparation comprises obtaining a biological sample comprising one or more biological entities, wherein the biological entities comprise RNA; lysing said one or more biological entities, thereby releasing a plurality of RNA molecules therefrom; and performing a reverse transcriptase reaction on the released RNA molecules to produce a plurality of DNA molecules, wherein said method extracts said nucleic acid molecules from said one or more biological entities within 5 minutes or less at a quality sufficient to successfully perform a polymerase chain reaction (PCR). In some cases, the method is performed by a handheld device. In some cases, a quality sufficient to successfully perform a polymerase chain reaction comprises nucleic acid molecules which amplify with at least 70% efficiency as determined by a qPCR standard curve. In some cases, the method produces at least 100 μL of a solution containing the nucleic acid molecules. In some cases, the method produces at least 300 of a solution containing the nucleic acid molecules. In some cases, the method produces at least 500 of a solution containing the nucleic acid molecules. In some cases, the method further comprises catching biological entities on a filter and subjecting the biological entities and filter to an air wash. In some cases, the biological entities are washed with a volume of air sufficient to dry the filter. In some cases, the biological entities are washed with at least about 1.5 mL of air.

In another aspect, a device is configured to perform a method as described herein, wherein said device comprises an input port, configured to receive said biological sample comprising one or more biological entities; a holding tank, operably coupled to said input port, an inactivation section, and containing a heating element; and an output port. In some cases, the device further comprises a permanent vent. In some cases, the holding tank further comprises an electrical probe which can sense the presence of liquid in the holding tank. In some cases, the inactivation chamber comprises a serpentine path.

In another aspect, a method of DNA preparation comprises conveying a biological sample comprising RNA into a sample input module of a molecular diagnostic test device; and actuating the molecular diagnostic test device to: lyse the biological sample in a lysing module, convey the biological sample from the lysing module to a reverse transcription module, the reverse transcription module including a heater and defining a first reaction volume and a second reaction volume, and further comprising lyophilized reagents for a reverse transcription reaction; maintain an input solution containing the biological sample and the reagents for reverse transcription within the first reaction module to reverse transcribe at least a portion of the biological sample thereby producing a plurality of DNA molecules; activate the heater to heat a portion of the lysing module to produce an inactivation temperature zone within the second reaction volume; and produce a flow of the input solution within the second reaction volume such that a volume of the input solution is heated within the inactivation temperature zone to inactivate an enzyme within the input solution. In some cases, the volume of the input solution is at least 10 microliters. In some cases, the volume of the input solution is produced within five minutes or less. In some cases, the second reaction volume is a serpentine flow path. In some cases, a wall of the lysing module that defines the second reaction volume has a surface area, a ratio of the surface area to the second reaction volume being greater than about 10 cm-1. In some cases, the volume of the input solution is heated to an inactivation temperature of between about 57 degrees Celsius and about 100 degrees Celsius for a time period from about 15 seconds. In some cases, the flow of the input solution is such that the volume of the input solution is heated to an inactivation temperature of between about 92 degrees Celsius and about 98 degrees Celsius for a time period of at least about 25 seconds. In some cases, the first reaction volume is in fluid communication with the second reaction volume; and the reverse transcription module defines a vent opening into the first reaction volume. In some cases, the volume of the input solution is heated to an inactivation temperature of at least about 95 degrees Celsius; and the input solution within the first reaction module contains at least one of a salt or a sugar formulated to raise a boiling temperature of the input solution. In some cases, the portion of the reverse transcription module is a second portion, the actuating the molecular diagnostic test device further causes the molecular diagnostic test device to: heat a first portion of the lysing module to produce a lysing temperature zone within the second reaction volume, the flow of the input solution within the second reaction volume being such that the volume of the input solution is heated within the lysing temperature zone to lyse a biological entity within the volume of the input solution. In some cases, the actuating the molecular diagnostic test device causes the molecular diagnostic test device to: convey the biological sample from the sample input module through a filter to retain a biological entity with the biological sample on the filter; and produce a flow of an elution buffer through the filter to produce the input solution and convey the input solution to the lysing module. In some cases, the actuating the molecular diagnostic test device includes moving a sample actuator to produce a pressure within the sample input module to convey the biological sample from the sample input module towards the lysing module. In some cases, the sample actuator is a non-electronic actuator. In some cases, the actuating the molecular diagnostic test device further causes the molecular diagnostic test device to: receive an electronic signal from a sensor within the reverse transcription module, the electronic signal indicating the presence of the input solution within the first reaction module; and activate the heater in response to the electronic signal. In some cases, the actuating the molecular diagnostic test device further causes the molecular diagnostic test device to: heat a portion of an amplification module within the molecular diagnostic test device to amplify a nucleic acid from the plurality of nucleic acid molecules to produce an output containing a target amplicon; and convey the output to a detection module of the molecular diagnostic test device. In some cases, the method further comprises viewing a visible signal indicating a presence of the target amplicon; and discarding, after the viewing, the molecular diagnostic test device.

In another aspect an apparatus, comprises a housing; a sample input module defining an input reservoir configured to receive a biological sample, the biological sample containing a biological entity; a lysing module disposed within the housing, the lysing module including a heater and first flow member, the first flow member defining a first volume and a second volume, the first volume configured to receive an input solution containing at least the biological sample and a lysis buffer, the heater coupled to the first flow member and configured to convey thermal energy into the second volume to A) lyse at least a portion of the biological sample thereby releasing a plurality of nucleic acid molecules and B) inactivate an enzyme within the input solution when a volume of the input solution flows through the second volume; a reverse transcription module disposed within the housing, the reverse transcription module including a heater and first flow member, the first flow member defining a first volume and a second volume, the first volume configured to receive an input solution containing at least the biological sample and a lysis buffer, the first volume further containing lyophilized reagents for a reverse transcription reaction, the heater coupled to the first flow member and configured to convey thermal energy into the second volume to A) reverse transcribe at least a portion of the biological sample thereby releasing a plurality of nucleic acid molecules and B) inactivate an enzyme within the input solution or within the lyophilized reverse transcription reagents when a volume of the input solution flows through the second volume; and an amplification module disposed within the housing, the amplification module including a second flow member configured to receive the volume of the input solution from the lysing module, the amplification module configured to amplify a nucleic acid molecule from the plurality of nucleic acid molecules within the volume of the input solution to produce an output containing a target amplicon. In some cases, the second volume of the reverse transcription module is a serpentine flow path. In some cases, a wall of the reverse transcription module that defines the second volume has a surface area, a ratio of the surface area to the second reaction volume being greater than about 10 cm-1. In some cases, the first volume is in fluid communication with the second reaction volume; and the reverse transcription module defines a vent opening into the first volume. In some cases, the lysing module includes a sensor disposed within the first volume, the sensor configured to produce an electronic signal indicating the presence of the input solution within the first module, the heater activated in response to the electronic signal. In some cases, the heater is a first heater; the second flow member defines an amplification flow path; and the amplification module includes a second heater different from the first heater, the second heater coupled to the second flow member and configured to convey thermal energy into the amplification flow path to amplify the nucleic acid molecule from the plurality of nucleic acid molecules. In some cases, the apparatus further comprises a non-electronic sample actuator to produce a pressure within the sample input module to convey the biological sample from the sample input module towards the lysing module; and a fluid pump disposed within the housing, the fluid pump configured to produce a flow of the input solution from the lysing module to the amplification module. In some cases, the flow of the input solution from the lysing module to the amplification module is in a first direction; and the lysing module includes a check valve to configured to prevent a flow of the input solution in a second direction. A device comprising a holding tank which contains two electrical probes which may be used to determine the electrical resistance of the fluid within the holding tank, thus determining whether liquid has entered the holding tank.

In another aspect, an apparatus comprises a reverse transcription module disposed within a molecular diagnostic test device, the reverse transcription module including a heater and a flow member, the flow member defining a first volume and a second volume, the first volume containing a lyophilized reverse transcriptase enzyme and configured to receive an input solution containing at least a biological sample, the heater coupled to the flow member and configured to convey thermal energy into the reverse transcription module to facilitate a thermal reaction on the input solution when a volume of the input solution flows through the second volume; and a sensor at least partially disposed within the first volume the sensor configured to produce a signal when the input solution is within the first volume, a portion of the molecular diagnostic test device being actuated in response to the signal. In some cases, the sensor includes a first electrode and a second electrode, the first electrode disposed within the first volume, the second electrode disposed within the second volume, spaced apart from the first electrode, the sensor configured to determine an electrical resistance of the input solution between the first electrode and the second electrode and produce the signal associated with the electrical resistance. In some cases, the heater is actuated in response to the signal. In some cases, the apparatus further comprises an amplification module disposed within the housing, the amplification module including an amplification flow member configured to receive the volume of the input solution from the reverse transcription module, the amplification module configured to amplify a nucleic acid molecule from a plurality of nucleic acid molecules within the volume of the input solution to produce an output containing a target amplicon, the amplification module being actuated in response to the signal. In another aspect, a method for increasing the concentration of a biological entity in a liquid comprises obtaining a plurality of hydrogel particles functionalized with affinity baits for said biological entity; incubating a first volume of the liquid containing the biological entity with the hydrogel particles; flowing the liquid containing the biological entity and the hydrogel particles through a filter with a pore size such that the hydrogel particles cannot pass through the filter; and eluting the hydrogel particles and bound biological entity from the filter in a second volume of liquid, wherein the second volume of liquid is smaller than the first volume of liquid, thus increasing the concentration of the biological entity in the liquid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts data generated from a real-time PCR reaction performed on DNA extracted from clinical samples utilizing the methods provided herein.

FIG. 2 depicts data generated from a real-time PCR reaction performed on DNA extracted from clinical samples utilizing standard DNA extraction methods.

FIG. 3 depicts a comparison of data generated from a real-time PCR reaction performed on DNA extracted from a clinical sample positive for both N. gonorrhoeae and C. trachomatis (Sample 122) and a clinical sample positive for N. gonorrhoeae (Sample 117) utilizing the methods provided herein versus standard DNA extraction methods.

FIG. 4 depicts a comparison of data generated from a real-time PCR reaction performed on DNA extracted from a clinical sample positive for both N. gonorrhoeae and C. trachomatis (Sample 122) and a clinical sample positive for N. gonorrhoeae (Sample 117) utilizing the methods provided herein versus standard DNA extraction methods.

FIG. 5 depicts a comparison of data generated from a real-time PCR reaction performed on DNA extracted from a clinical sample positive for both N. gonorrhoeae and C. trachomatis (Sample 122), a clinical samples positive for C. trachomatis (Samples 101 and 108) utilizing the methods provided herein versus standard DNA extraction methods.

FIG. 6 depicts a comparison of data generated from a real-time PCR reaction performed on DNA extracted from a clinical sample positive for both N. gonorrhoeae and C. trachomatis (Sample 122) and clinical samples positive for C. trachomatis (Samples 101 and 108) utilizing the methods provided herein versus standard DNA extraction methods.

FIG. 7 depicts a comparison of data generated from a real-time PCR reaction performed on N. gonorrhoeae DNA utilizing different sets of primers.

FIG. 8 depicts a comparison of data generated from a real-time PCR reaction performed on C. trachomatis DNA utilizing different sets of primers.

FIG. 9 depicts data generated from a real-time PCR reaction performed on N. gonorrhoeae DNA spiked into a sample and PCR mixture to test for sample inhibition.

FIG. 10 is a schematic illustration of a molecular diagnostic test device, according to an embodiment, which can perform the methods described herein.

FIG. 11 is an exploded view of a molecular diagnostic test device, according to an embodiment, which can perform the methods described herein.

FIG. 12 depicts an example of a sample preparation device amenable to performing the methods as described herein.

FIG. 13 is a perspective view of a lysing module according to an embodiment, which is amenable to performing the methods as described herein.

FIG. 14 is an exploded view of the lysing module shown in FIG. 13.

FIG. 15 is a top view of a portion of the lysing module shown in FIG. 13.

FIG. 16 is a cross-sectional view of the lysing module shown in FIG. 13.

FIGS. 17 and 18 is are perspective views of a lysing module according to an embodiment, which can perform any of the methods described herein.

FIG. 19 is a bottom view of the lysing module shown in FIGS. 17 and 18.

FIG. 20 is a cross-sectional view of the lysing module shown in FIGS. 17 and 18 taken along line X₁-X₁ in FIG. 19.

FIG. 21 is a cross-sectional view of the lysing module shown in FIGS. 17 and 18 taken along line X₂-X₂ in FIG. 19.

FIG. 22 is a perspective view of a portion of the lysing module shown in FIGS. 17 and 18.

FIG. 23 is a schematic illustration of a portion of a molecular diagnostic test device, according to an embodiment, which can perform the methods described herein.

FIG. 24 is a schematic illustration of a molecular diagnostic test device, according to an embodiment, which can perform the methods described herein.

FIG. 25 illustrates the results of a PCR reaction performed upon DNA extracted using the methods of this disclosure.

FIG. 26 illustrates the results of a PCR reaction performed upon DNA extracted using the methods of this disclosure.

FIG. 27 illustrates the results of a PCR reaction performed upon DNA extracted using the methods of this disclosure.

FIG. 28 illustrates a block diagram of a device including a reverse transcription module.

FIG. 29 illustrates a temperature profile in a reverse transcription module.

FIG. 30 illustrates a possible chamber design for a reverse transcription module.

FIG. 31 illustrates the bottom view of a possible chamber design for a reverse transcription module.

FIG. 32 illustrates an example of a functionalized nanoparticle.

FIG. 33 illustrates a proposed model of functionalized nanoparticle binding to viruses.

FIG. 34 illustrates a block diagram of a device including a reverse transcription module.

FIG. 35 is a schematic illustration of a portion of a molecular diagnostic test device, according to an embodiment, which can perform the methods described herein.

FIG. 36 illustrates capture of viral nucleic acid with affinity particles

FIG. 37 illustrates capture of infectious viral particles with affinity particles.

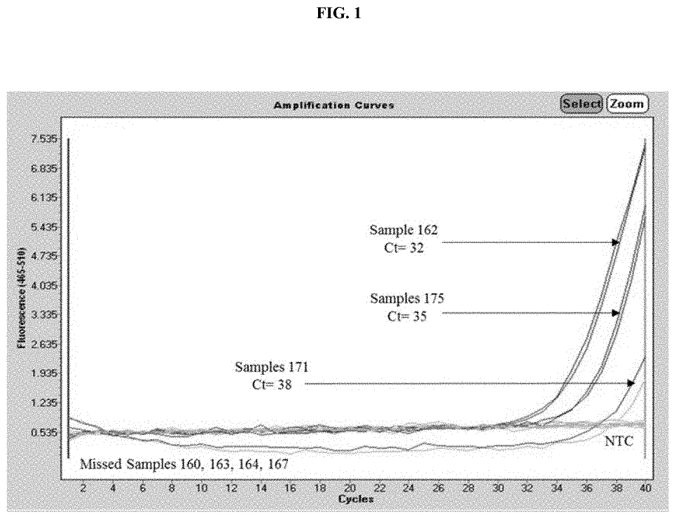

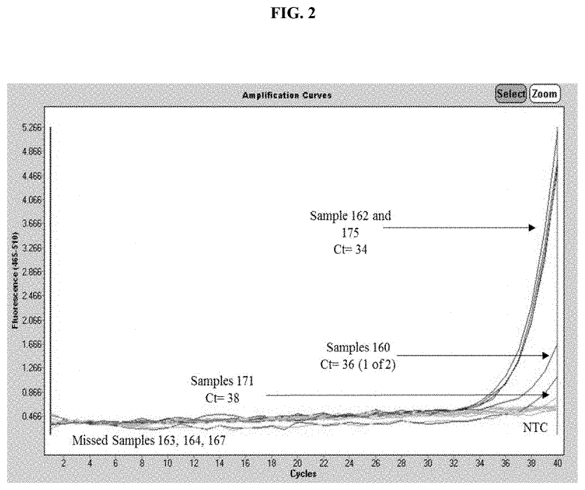

### DETAILED DESCRIPTION OF THE INVENTION

Disclosed herein are devices and methods for the preparation of nucleic acid molecules for downstream applications. In some cases, the devices and methods are utilized for the extraction of nucleic acid molecules from a biological sample. In some cases, the devices and methods are utilized for the purification of nucleic acid molecules from a biological sample. In some cases, the devices and methods are utilized to produce and detect a cDNA from an RNA isolated from a biological sample. The devices described herein may include self-contained, handheld devices. The devices described herein may include one or more components that aid in the extraction, purification, and/or processing of a biological sample and the nucleic acids contained therein. In some cases, the methods include the use of a device that includes one or more components that aid in the extraction, purification, and/or processing of a biological sample and the nucleic acids contained therein. In some cases, the processing of a biological sample may include a reverse transcription step which may be achieved by a reverse transcriptase.

In one aspect, a method is provided for nucleic acid extraction. The method may include one or more steps including: (a) obtaining a biological sample comprising one or more biological entities; (b) capturing the one or more biological entities on a filter; (b) washing the filter with a wash solution and/or air; (c) eluting the one or more biological entities from the filter; and (d) lysing the one or more biological entities, thereby releasing a plurality of nucleic acid molecules therefrom. In some cases, the wash solution comprises bovine serum albumin and/or a detergent. In some cases, the wash solution comprises about 0.1% to 5% bovine serum albumin. In some cases, the wash solution comprises about 0.1%, 0.2%, 0.3%, 0.4%, 0.5%, 1%, 1.5%, 2%, 2.5%, 3%, 4%, or 5% bovine serum albumin. In some cases, the wash solution comprises about 0.1% to 20% detergent. In some cases, the wash solution comprises about 1%, 2%, 3%, 4%, 5%, 6%, 7%, 8%, 9%, or 10% detergent. In some cases, the detergent is Tween-20. In some embodiments the method may not require use of a filter. In other embodiments the method may use a filter but not require a wash solution. In some cases, the method further includes a step of reverse transcribing an RNA molecule to produce a cDNA molecule. In some further cases, the method includes a preliminary step for increasing the concentration of one or more biological entities in the sample. This step may involve the use of affinity beads designed to bind to pathogens or analytes in the sample. The affinity beads may be nanoparticles or microparticles, (functionalized nanoparticles or functionalized microparticles).

In some cases, the method involves obtaining or providing a biological sample. The biological sample can be derived from a non-cellular entity comprising polynucleotides (e.g., a virus) or from a cell-based organism (e.g., member of archaea, bacteria, or eukarya domains).

Generally, the biological sample will contain one or more biological entities that comprise one or more polynucleotides or nucleic acid molecules. A “nucleic acid molecule”, “nucleic acid” or “polynucleotide” may be used interchangeably throughout and may refer to deoxyribonucleic acid (DNA) or ribonucleic acid (RNA) including known analogs or a combination thereof unless otherwise indicated. Nucleic acid molecules to be profiled herein can be obtained from any source of nucleic acid. The nucleic acid molecule can be single-stranded or double-stranded. In some cases, the nucleic acid molecules are RNA. RNA can include, but is not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long nc RNAs. In some cases, the nucleic acid molecules are DNA. The DNA can be mitochondrial DNA, complementary DNA (cDNA), or genomic DNA. In some cases, the nucleic acid molecules are genomic DNA (gDNA). The DNA can be plasmid DNA, cosmid DNA, bacterial artificial chromosome (BAC), or yeast artificial chromosome (YAC). The DNA can be derived from one or more chromosomes. For example, if the DNA is from a human, the DNA can derived from one or more of chromosomes 1, 2, 3, 4, 5, 6, 7, 8, 9, 10, 11, 12, 13, 14, 15, 16, 17, 18, 19, 20, 21, 22, X, or Y. The source of nucleic acid for use in the methods and compositions described herein can be a sample comprising the nucleic acid.

### Concentrating One or More Biological Entities in the Sample

In some aspects, the methods involve capturing one or more biological cells or biological entities (e.g., a virus) with a capture particle or affinity bead. Various methodologies may be used to capture and concentrate pathogens from biological fluids (e.g., blood, plasma, homogenized tissue, urine). The capture methods may be generic and bind to any cells or biological entities in a sample, or may be specific to a class or type of biological entity. In other cases, the capture methods may be specific to a family of pathogens, for example a family of bacteria or viruses. In some cases, the capture methods may be specific to a single species of pathogen, for example a single species of bacteria or virus. In some cases the capture methods may be designed to bind to several related or unrelated pathogens. For example the capture methods may be designed to bind one or more of the following pathogens: Ebola virus, Sudan virus, Tai Forest virus, Bundibugyo virus, Yersinia pestis, Zika virus, Plasmodium falciparum, Leptospira interrogans, Dengue virus, Chikungunya virus, Crimean-Congo hemorrhagic fever virus, and Lassa virus.

In some cases, the capturing and concentration of a biological entity is achieved by use of a particle which the biological entity adheres to. The particle may be made of any substance. In some embodiments, the particle is a hydrogel particle. In some examples the particle is a hydrogel particle based on cross-linked N-isopropylacrylamide (NIPAm). The particle may comprise a core with a porous coating. An example of such a particle is shown in FIG. 33. In some cases, the particle may have a porous coating which performs a size exclusion function limiting the biological entities which may bind the particle.

The particle may be functionalized with a variety of affinity baits to facilitate the binding and retention of biological targets. In some cases, the functionalized particle may be composed of a core containing high affinity aromatic baits, surrounded by a sieving shell. Examples of aromatic baits include: Cibacron Blue, Allylamine and Methacrylate. The outer shell may be tailored for active exclusion of high abundance proteins. For example, the outer shell may contain vinyl sulfonic acid for active molecular sieving of high-abundance proteins. The functionalized particles may be tailored to capture target analytes from a variety of complex biological matrices, including blood, serum, plasma, saliva and nasopharyngeal fluids. The target analytes may be proteins, nucleic acids, viruses or bacteria. The functionalized particles may capture live bacteria and intact viruses without causing damage.

The functionalized particles may be nanoparticles. In some cases the functionalized nanoparticles have an average diameter of about 10-100, 20-40, 30-50 or 20-30 nm. In some embodiments, a functionalized nanoparticle may have a diameter of about 1, 2, 3, 4, 5, 6, 7, 8, 9, 10, 11, 12, 13, 14, 15, 16, 17, 18, 19, 20, 25, 30, 35, 40, 45, 50 or more than 50 nm In some embodiments, the functionalized particles may be microparticles. In some cases the functionalized microparticles have an average diameter of about 10-100, 20-40, 30-50 or 20-30 μm.

In some instances, a functionalized microparticle may be created by attaching one or more functionalized nanoparticles to a larger particle. The larger particle may have a diameter of about 1, 2, 3, 4, 5, 6, 7, 8, 9, 10, 11, 12, 13, 14, 15, 16, 17, 18, 19, 20, 25, 30, 35, 40, 45, 50 or more than 50 μm. In some cases the larger particle may have a diameter between about 1 and 10, 1 and 5, 5 and 10, 3 and 8, or 2 and 7 μm. The larger particle may be a hydrogel particle or a different type of particle. In some cases, the larger particle is a polystyrene particle. A larger particle may be bound to an average of 1, 2, 3, 4, 5, 6, 7, 8, 9, 10, 11, 12, 13, 14, 15, 16, 17, 18, 19, 20, 30, 40, 50, or more than 50 functionalized nanoparticles. The functionalized nanoparticles may be covalently bound to the larger particle. In some cases, the functionalized nanoparticle chemistry may incorporate amine containing monomers into the hydrogel matrix.

To concentrate a biological entity within a sample one or more functionalized nanoparticles or functionalized microparticles designed to bind said biological entity may be added to the sample. After incubation of the functionalized nanoparticles or functionalized microparticles in the sample for a sufficient time at a suitable temperature to allow binding of the biological entity, the functionalized nanoparticles or functionalized microparticles are extracted from the sample. In some cases, the functionalized nanoparticles or functionalized microparticles are extracted by flowing the sample through a filter with a pore size smaller than the size of the particles. The functionalized nanoparticles or functionalized microparticles and associated biological entities may subsequently be washed off the filter and nucleic acids may be released by lysis. In some embodiments functionalized nanoparticles or functionalized microparticles are added to a sample prior to processing the sample through a method of device as described herein. In some embodiments, functionalized microparticles will be lyophilized and put into sample collection tubes, so upon collection of a sample into the tube, the functionalized microparticles will hydrate and actively capture the relevant biological entities. The sample and functionalized microparticle mixture may be used directly in the methods and devices described herein.

The incubation step for the functionalized microparticles and the biological entities may be at least about 1, 2, 3, 4, 5, 6, 7, 8, 9, 10, 11, 12, 13, 14, 15, 16, 17, 18, 19, 20, 25, 30, 35, 40, 45, 50, 55, or 60 minutes. In some cases the incubation step is between 1 and 60, 1 and 30, 1 and 20, 1 and 15, 1 and 10 or 1 and 5 minutes. In some cases the incubation step is less than 1 minute. In some cases, the incubation step is performed at room temperature. In some cases, the incubation step is performed at a temperature between about 15 and 80, 20 and 40, 20 and 30, 20 and 25, or 25 and 30° C.

The functionalized nanoparticles or functionalized microparticles may provide an enrichment of a biological entity in a solution by about 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 fold. Using a method or device as described herein with a functionalized microparticle may result in an increase in the amount of nucleic acid extracted or prepared of about 2, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 fold compared to the same method or device without the functionalized microparticle.

### Filter

In some aspects, the methods involve capturing one or more biological cells or biological entities (e.g., a virus, or a functionalized microparticle with trapped virus particles) present in the biological sample on a filter membrane. The filter membrane may be of any suitable material, non-limiting examples including nylon, cellulose, polyethersulfone (PES), polyvinylidene difluoride (PVDF), polycarbonate, borosilicate glass fiber and the like. In some examples, the filter membrane is nylon. In some cases, the filter membrane has an average pore size of about 0.2 μm to about 20 μm. For example, the filter membrane may have an average pore size of about 0.2 μm, about 0.5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, or greater than 20 μm. In some examples, the surface of the filter membrane may be chemically treated or coated in such a way as to improve the binding of a biological cell or entity to the membrane. For example, without limitation, the filter membrane may be treated with sodium polyphosphate.

Clinical swab samples may contain mucus (or other substances) which can lead to clogging of the filter used in sample prep. If the filter is clogged then pressures may build up which may lead to leaks in the fluidic path of the sample prep device and/or tears or breaks in the capture filter itself. In some examples a second filter may be provided which sits next to a first filter. For example, a mesh screen may be placed on the input side of the 5 micron nylon filter. This may reduce pressure from mucus samples and also prevent the 5 micron nylon filter from breaking. A mesh screen could also be placed on the exit side of the 5 micron nylon filter which would also prevent the 5 micron nylon filter from breaking, however this may not reduce the pressure required to push a sample (mucus) through.

The mesh screen may be made from any plastic materials and may contain pore sizes from 1 micron to 1000 microns. In some embodiments the mesh screen may be a woven nylon mesh with 100 micron pores. The mesh screen is assembled into the housing that also contains the 5 micron nylon filter. The second filter may have a much larger pore size than the first filter and prevent clogging of the first filter. For example the first filter may have a pore size of about 0.1-20, 1-15, 1-10, 5-10, 1-5 or 0.1-1 μm while the second filter has a pore size of about 10-1000, 50-500, 100-500, 50-100, or 100-200 μm. In one example the first filter has a pore size of 5 μm and the second filter has a pore size of 100 μm. The mesh filter may also be made from non-woven polypropylene. The mesh screen may have a thickness of about 150 μm, 200 μm or greater than 200 μm. After the biological cells or biological entities are captured on the filter membrane, the filter membrane may be optionally washed with one or more wash steps. The wash step may be utilized to, for example, remove any undesired material from the membrane. In some cases, the wash step may involve pushing or forcing a fluid solution over or through the membrane (e.g., a buffer). The volume of wash solution may be from about 10 μL to about 50 mL. For example, the volume of wash solution may be about 10 μL, about 50 μL, about 100 μL, about 200 μL, about 300 μL, about 400 μL, about 500 μL, about 600 μL, about 700 μL, about 800 μL, about 900 μL, about 1 mL, about 5 mL, about 10 mL, about 15 mL, about 20 mL, about 25 mL, about 30 mL, about 35 mL, about 40 mL, about 45 mL, about 50 mL or greater than 50 mL. In other cases, the wash step may involve pushing or forcing air over or through the membrane. This step may be advantageous in decreasing the volume of sample buffer that is carried over into the lysis buffer. The volume of air wash may be from about 0.1 μL to about 100 L, or about 10 μL to about 50 mL. For example, the volume of air wash may be about 10 about 50 about 100 about 200 about 300 about 400 about 500 about 600 about 700 about 800 about 900 about 1 mL, about 5 mL, about 10 mL, about 15 mL, about 20 mL, about 25 mL, about 30 mL, about 35 mL, about 40 mL, about 45 mL, about 50 mL or greater than 50 mL. In some cases, an air wash volume of about 1-5 mL may be preferred, For example an air wash may be have a volume of about 1.5 mL. In cases where an air wash is used the subsequent liquid wash may be more effective and/or the final eluted sample may be cleaner than if no air wash were used. In some cases, the wash step involves both a fluid wash step and an air wash step, performed in any order. In some cases, the wash solution comprises bovine serum albumin and/or a detergent. In some cases, the wash solution comprises about 0.1% to 5% bovine serum albumin. In some cases, the wash solution comprises about 0.1%, 0.2%, 0.3%, 0.4%, 0.5%, 1%, 1.5%, 2%, 2.5%, 3%, 4%, or 5% bovine serum albumin. In some cases, the wash solution comprises about 0.1% to 20% detergent. In some cases, the wash solution comprises about 1%, 2%, 3%, 4%, 5%, 6%, 7%, 8%, 9%, or 10% detergent. In some cases, the detergent is Tween-20. In some embodiments, the bovine serum albumin and/or detergent increase the viscosity of the wash solution in manner which increases the surface area of the filter contacted with the wash solution during a wash step as compared to a wash solution lacking one or both of bovine serum albumin and detergent.

After the membrane is washed, the biological cells or entities captured on the membrane may be lysed or otherwise disrupted so as to release a plurality of nucleic acid molecules contained therein. The methods and devices of this disclosure may use chemical, enzymatic and/or thermal methods to lyse the sample. In some embodiments the methods and devices of this disclosure do not use ultrasound to lyse the sample. In some cases, the cells may be lysed by heating the sample. For example the sample may be heated to greater than about 90° C. for longer than about 10 seconds. In some examples heating the sample to about 95° C. for about 20 seconds is seen to be sufficient to lyse the sample.

In some cases, lysis involves flowing a lysis buffer over the biological cells or entities captured on the membrane. In some cases, the lysis buffer is flowed through the filter membrane. In other cases, the lysis buffer is back-flowed through the filter membrane. The lysis buffer may be osmotically imbalanced so as to force fluid into the cells to rupture the cell membranes. In some cases, the lysis buffer may include one or more surfactants or detergents. Non-limiting examples of surfactants or detergents that may be used include: nonionic surfactants including polyoxyethylene glycol alkyl ethers (sold as Brij® series detergents including Brij® 58, Brij® 52, Brij® L4 and Brij® L23), octaethylene glycol monododecyl ether, pentaethylene glycol monododecyl ether, polyoxypropylene glycol alkyl ethers, glucoside alkyl ethers (e.g., decyl glucoside, lauryl glucoside, octyl glucoside), polyoxyethylene glycol octylphenol ethers (e.g., Triton X-100), polyoxyethylene glycol alkylphenol ethers (e.g., nonoxynol-9), glycerol alkyl esters (e.g., glyceryl laurate), polyoxyethylene glycol sorbitan alkyl esters (e.g., polyoxyethylene glycol (20) sorbitan monolaurate, polyoxyethylene glycol (40) sorbitan monolaurate, polyoxyethylene glycol (20) sorbitan monopalmitate, polyoxyethylene glycol (20) sorbitan monostearate, polyoxyethylene glycol (4) sorbitan monostearate, polyoxyethylene glycol (20) sorbitan tristearate, polyoxyethylene glycol (20) sorbitan monooleate)), sorbitan alkyl esters (e.g., sorbitan monolaurate, sorbitan monopalmitate, sorbitan monostearate, sorbitan monooleate, sorbitan sesquioleate, sorbitan trioleate, sorbitan isostearate), cocamide monoethanolamine, cocamide diethanolamine, dodecyldimethylamine oxide, poloxamers including those sold under the Pluronic®, Synperonic® and Kolliphor® tradenames, and polyethoxylated tallow amine (POEA); anionic surfactants including ammonium lauryl sulfate, ammonium perfluorononanoate, docusate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium lauryl ether sulfate, sodium lauroyl sarcosinate, sodium myreth sulfate, sodium pareth sulfate, sodium stearate; cationic surfactants including benzalkonium chloride, benzethonium chloride, bronidox, cetrimonium bromide, cetrimonium chloride, distearyldimethylammonium chloride, lauryl methyl gluceth-10 hydroxypropyl dimonium chloride, octenidine dihydrochloride, olaflur, and tetramethylammonium hydroxide; and Zwitterionic surfactants including CHAPS detergent, cocamidopropyl betaine, cocamidopropyl hydroxysultaine, dipalmitoylphosphatidylcholine, lecithin, hydroxysultaine, and sodium lauroamphoacetate.

In some cases, the lysis buffer may contain an antifoaming agent for preventing or minimizing foaming. Non-limiting examples of antifoaming agents include Antifoam SE-15, Antifoam 204, Antifoam Y-30. In some cases, the lysis buffer may contain a preservative, for example an antimicrobial agent. Non-limiting examples of antimicrobials may include ProClin™ 150, ProClin™ 200, ProClin™ 300, and ProClin™ 950.

In cases where the desired nucleic acid molecules are RNA, the lysis buffer may include one or more agents that prevent degradation of the RNA, such as, for example, an RNAse inhibitor. The volume of lysis buffer flowed over the membrane can be from about 10 μL to about 50 mL. For example, the volume of lysis buffer may be about 10 μL, about 50 μL, about 100 μL, about 200 μL, about 300 μL, about 400 μL, about 500 μL, about 600 μL, about 700 μL, about 800 μL, about 900 μL, about 1 mL, about 5 mL, about 10 mL, about 15 mL, about 20 mL, about 25 mL, about 30 mL, about 35 mL, about 40 mL, about 45 mL, about 50 mL or greater than 50 mL.

In some cases, the lysis buffer contains one or more enzymes. In some cases, the one or more enzymes comprise Proteinase K. Proteinase K may be present in the lysis buffer at a concentration of about 0.001 mg/mL to about 10 mg/mL. For example, the concentration of proteinase K in the lysis buffer may be about 0.001 mg/mL, about 0.005 mg/mL, about 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL or greater than about 10 mg/mL. In some cases, the one or more enzymes comprise lysozyme to process gram-positive organisms. Lysozyme may be present in the lysis buffer at a concentration of about 0.001 mg/mL to about 10 mg/mL. For example, the concentration of lysozyme in the lysis buffer may be about 0.001 mg/mL, about 0.005 mg/mL, about 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL or greater than about 10 mg/mL. In some cases, the one or more enzymes comprise zymolyase to process yeast. Zymolase may be present in the lysis buffer at a concentration of about 0.001 mg/mL to about 10 mg/mL. For example, the concentration of zymolase in the lysis buffer may be about 0.001 mg/mL, about 0.005 mg/mL, about 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL or greater than about 10 mg/mL. Additional enzymes that may be used include, without limitation, lyticase, chitinase or gluculase, for e.g., the extraction of nucleic acids from yeast. In some examples, if more than one lysis enzyme is used, the enzymes may be added in sequence. For example, lysozyme may be added first, followed by an incubation period, and subsequently followed by addition of proteinase K and an additional incubation period. In some cases, the lysis buffer does not contain any enzymes.

In some aspects, the methods may involve one or more incubation steps. The one or more incubation steps may be performed in the lysis buffer in order to ensure complete lysis or disruption of the biological cell or entity and/or to destroy any inhibitory protein that may be present. The incubation step may involve holding the biological cell or entity in the lysis buffer for a period of time. In some cases, the incubation step involves holding the biological cell or entity in the lysis buffer for a period of time at a specified temperature. In a non-limiting example, the biological cell or entity is incubated in the lysis buffer from about 0.01 seconds to about 48 hours. For example, the biological cell or entity is incubated in the lysis buffer from about 0.01 seconds, about 0.05 seconds, about 1 second, about 10 seconds, about 30 seconds, about 1 minute, about 5 minutes, about 10 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 48 hours, or greater than 48 hours. In some examples, the biological cell or entity is incubated in the lysis buffer at a specified temperature, for example, from about 4° C. to about 75° C. For example, the biological cell or entity is incubated in the lysis buffer at a temperature of about 4° C., about 10° C., about 15° C., about 20° C., about 25° C., about 30° C., about 40° C., about 45° C., about 50° C., about 55° C., about 60° C., about 65° C., about 70° C., about 75° C. or greater than 75° C. Generally, the temperature conditions will be selected so as to promote disruption of the biological cell or entity. For example, if the lysis buffer contains an enzyme (e.g., Proteinase K), the temperature may be selected such that the enzyme retains catalytic activity. In some cases, the temperature may be selected for optimal catalytic activity of the lysis enzyme. The temperature may also be selected to neutralize any inhibitory proteins within the sample, but should not destroy or disrupt the integrity of the nucleic acid molecules released therefrom. In some cases, the lysis buffer does not contain any enzymes.

The presence of one or more components (e.g., Proteinase K) in the lysis buffer may affect or interfere with downstream applications. In some cases, an additional incubation step may be performed to, for example, destroy or inactivate the one or more interfering components (e.g., Proteinase K) used in the lysis step. The subsequent incubation step may be from about 0.01 seconds to about 48 hours. For example, the biological cell or entity is incubated in the lysis buffer from about 0.01 seconds, about 0.05 seconds, about 1 second, about 10 seconds, about 30 seconds, about 1 minute, about 5 minutes, about 10 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 48 hours, or greater than 48 hours. In some examples, the additional incubation step may occur at a temperature between about 57° C. and about 100° C. For example, the additional incubation step may occur at a temperature of about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C.

In some aspects, the extracted nucleic acids may be utilized at this stage for any downstream processes, without any purification steps. In some cases, the extracted nucleic acid molecules may be used in one or more amplification reactions. For example, the extracted nucleic acid molecules may be used in one or more polymerase chain reactions (PCR). Any known method of PCR may be performed using the extracted nucleic acid molecules provided herein.

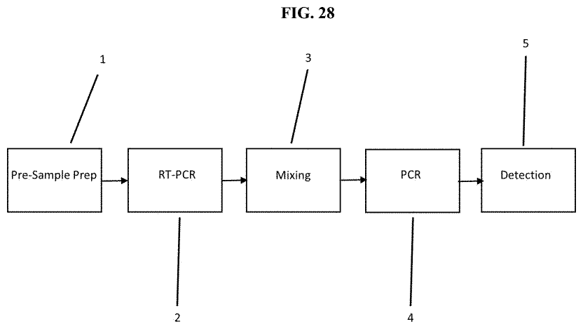

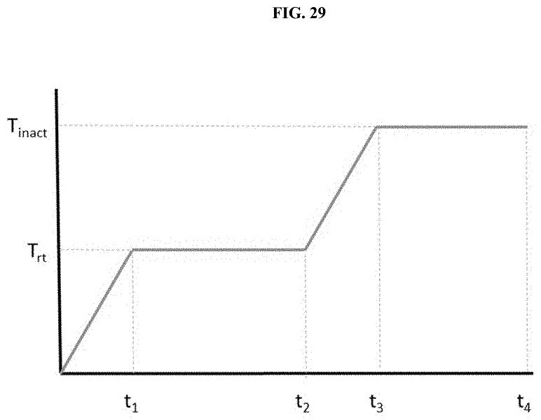

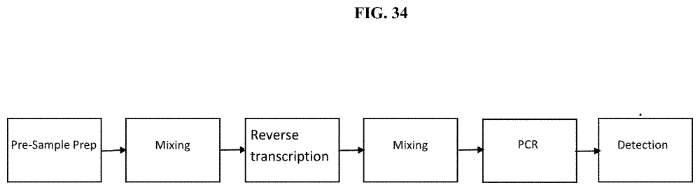

In some cases, when RNA is extracted, the RNA may be reverse transcribed (i.e., using a reverse transcriptase) prior to performing the downstream application. Briefly this may occur as in the diagram in FIG. 28, the sample is processed in a pre-sample prep stage which may include concentration, purification and lysis of the sample, the sample then moves to a RT-PCR step in which RNA molecules are reverse transcribed to DNA molecules, these move to a mixing compartment and thence to a PCR module and a detection module. Optionally this may occur as in FIG. 34 which includes an additional step between the pre-sample prep stage and the RT-PCR step in which the sample is mixed with reagents for performing the reverse transcriptase reaction. In some embodiments the steps of reverse transcription and PCR may occur in the same module, in this case the amplification module. Extracted RNA molecules may be incubated with one or more reverse transcriptase enzymes at a suitable temperature for reverse transcription to occur. The reverse transcriptase enzyme may be provided alone or with a buffer suitable for the reverse transcriptase reaction. The reverse transcriptase may be provided with a concentrated buffer designed to adjust the conditions of the extracted nucleic acid solution. In other cases no additional components are provided and the lysis buffer is suitable for reverse transcriptase. The incubation step may involve holding the biological cell or entity in the buffer for a period of time. In some cases, the incubation step involves holding the RNA molecule in the buffer for a period of time at a specified temperature. In a non-limiting example, the RNA molecule is incubated in the buffer from about 0.01 seconds to about 48 hours. For example, the RNA molecule is incubated in the buffer from about 0.01 seconds, about 0.05 seconds, about 1 second, about 10 seconds, about 30 seconds, about 1 minute, about 5 minutes, about 10 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 48 hours, or greater than 48 hours. In some examples, the RNA molecule is incubated in the buffer at a specified temperature, for example, from about 4° C. to about 75° C. For example, the RNA molecule is incubated in the buffer at a temperature of about 4° C., about 10° C., about 15° C., about 20° C., about 25° C., about 30° C., about 37, about 40° C., about 45° C., about 50° C., about 55° C., about 60° C., about 65° C., about 70° C., about 75° C. or greater than 75° C. Generally, the temperature conditions will be selected so as to promote activity of the reverse transcriptase enzyme. An example of a temperature profile for the reverse transcription reaction and inactivation step is shown by FIG. 29. The temperature of the RNA containing sample is heated to a temperature suitable for the RT reaction (T_{RT}). The temperature T_{RT} is reached by a first time (t1) and maintained for a period of time suitable to complete the reaction (t₁ to t₂). In the next stage from time t₂ to time t₃ the sample is heated to a temperature sufficient to inactivate the RT enzyme (T_{inact}). The sample is maintained at this temperature from time t₃ to time t₄, which provides a suitable amount of time to inactive the RT enzyme at a temperature of T_{inact}.

The presence of the reverse transcriptase in the buffer may affect or interfere with downstream applications. In some cases, an additional incubation step may be performed to, for example, destroy or inactivate the reverse transcriptase enzyme. The subsequent incubation step may be from about 0.01 seconds to about 48 hours. For example, the mixture of RNA and DNA molecules produced by the reverse transcriptase step is incubated from about 0.01 seconds, about 0.05 seconds, about 1 second, about 10 seconds, about 30 seconds, about 1 minute, about 5 minutes, about 10 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 48 hours, or greater than 48 hours. In some examples, the additional incubation step may occur at a temperature between about 57° C. and about 100° C. For example, the additional incubation step may occur at a temperature of about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C.

Biological Samples

In some cases, the biological sample can be a tissue sample. In some cases, the tissue sample is a blood sample. In some cases, the biological sample comprises a bodily fluid taken from a subject. In some cases, the bodily fluid comprises one or more cells comprising nucleic acids. In some cases, the one or more cells comprise one or more microbial cells, including, but not limited to, bacteria, archaebacteria, protists, and fungi. In some cases, the biological sample includes one or more virus particles. In some cases, the biological sample includes one or more RNA based virus particles. In some cases, the biological sample comprises one or more microbes that causes a sexually-transmitted disease. A sample may comprise a sample from a subject, such as whole blood; blood products; red blood cells; white blood cells; buffy coat; swabs; urine; sputum; saliva; semen; lymphatic fluid; endolymph; perilymph; gastric juice; bile; mucus; sebum; sweat; tears; vaginal secretion; vomit; feces; breast milk; cerumen; amniotic fluid; cerebrospinal fluid; peritoneal effusions; pleural effusions; biopsy samples; fluid from cysts; synovial fluid; vitreous humor; aqueous humor; bursa fluid; eye washes; eye aspirates; plasma; serum; pulmonary lavage; lung aspirates; animal, including human, tissues, including but not limited to, liver, spleen, kidney, lung, intestine, brain, heart, muscle, pancreas, cell cultures, as well as lysates, extracts, or materials and fractions obtained from the samples described above or any cells and microorganisms and viruses that may be present on or in a sample. A sample may comprise cells of a primary culture or a cell line. Examples of cell lines include, but are not limited to 293-T human kidney cells, A2870 human ovary cells, A431 human epithelium, B35 rat neuroblastoma cells, BHK-21 hamster kidney cells, BR293 human breast cells, CHO Chinese hamster ovary cells, CORL23 human lung cells, HeLa cells, or Jurkat cells. The sample may comprise a homogeneous or mixed population of microbes, including one or more of viruses, bacteria, protists, monerans, chromalveolata, archaea, or fungi. The biological sample can be a urine sample, a vaginal swab, a cervical swab, an anal swab, or a cheek swab. The biological sample can be obtained from a hospital, laboratory, clinical or medical laboratory. The sample can be obtained from a subject.

Non-limiting examples of sample sources include environmental sources, industrial sources, one or more subjects, and one or more populations of microbes. Examples of environmental sources include, but are not limited to agricultural fields, lakes, rivers, water reservoirs, air vents, walls, roofs, soil samples, plants, and swimming pools. Examples of industrial sources include, but are not limited to clean rooms, hospitals, food processing areas, food production areas, food stuffs, medical laboratories, pharmacies, and pharmaceutical compounding centers. Examples of subjects from which polynucleotides may be isolated include multicellular organisms, such as fish, amphibians, reptiles, birds, and mammals. Examples of mammals include primates (e.g., apes, monkeys, gorillas), rodents (e.g., mice, rats), cows, pigs, sheep, horses, dogs, cats, or rabbits. In some examples, the mammal is a human. In some cases, the sample is from an individual subject.

In some cases, the biological sample is provided in a sample buffer. In some cases, the sample buffer comprises bovine serum albumin and/or a detergent. In some cases, the sample buffer comprises about 0.1% to 5% bovine serum albumin. In some cases, the sample buffer comprises about 0.1%, 0.2%, 0.3%, 0.4%, 0.5%, 1%, 1.5%, 2%, 2.5%, 3%, 4%, or 5% bovine serum albumin. In some cases, the sample buffer comprises about 0.1% to 20% detergent. In some cases, the sample buffer comprises about 1%, 2%, 3%, 4%, 5%, 6%, 7%, 8%, 9%, or 10% detergent. In some cases, the detergent is Tween-20. The choice of sample buffer to be used may depend on the intended method. For example the choice of sample buffer may different when a wash step will be used to when a wash step is not used. If a wash step will not be used then the sample buffer may be a buffer suitable for lysis and subsequent PCR reactions.

Some commercial collection mediums or sample buffers contain chemicals for the preservation of microorganisms for future growth, or chemicals that lyse target organisms such as guanidinium thiocyanate. As such, these collection media are inhibitory to DNA polymerase and must be washed from a sample before PCR via filtration or similar process. The methods described herein may not require the target organism to be kept in a viable state, or for the sample buffer to be able to lyse the cells. Some components which may be found in a sample buffer suitable for use with the methods and devices of this disclosure include: Tris HCL, Tween-80, BSA, Proclin and Antifoam SE-15. In one embodiment a sample buffer may have a composition of: 50 mM Tris pH 8.4, Tween-80, 2% (w/v), BSA, 0.25% (w/v), Proclin 300, 0.03% (w/v), and Antifoam SE-15, 0.002% (v/v) made up in purified water.

Tris HCL is a common buffer for PCR. When it is heated during thermocycling, the pH may drop, for example a Tris buffer with pH of 8.4 at a temperature of 25° C. may drop to a pH of about ˜7.4 when heated to about 95° C. The range of concentrations could be from 0.1 mM to 1 M. The pH range could be from 6 to 10. Any other PCR compatible buffer could be used, for example HEPES.

Tween-80 is a nonionic surfactant and emulsifier that may help to elute target organisms off of a swab. The range of concentrations could be from 0.01% (w/v) to 20% (w/v). Any other PCR compatible surfactant and/or emulsifier could be used.

Proclin 300 is a broad spectrum antimicrobial used as a preservative to ensure a long shelf life of the collection media. It could be used from 0.01% (w/v) to 0.1% (w/v). Many other antimicrobials are known in the art and could be used in a sample buffer.

Antifoam SE-15 is present to reduce foaming during manufacturing and fluidic movement through the device. It could be used from 0.001% (v/v) to 1% (v/v). Any other antifoam agent could also be used, for example Antifoam 204, Antifoam A, Antifoam B, Antifoam C, or Antifoam Y-30.

The devices and methods provided herein may be utilized to prepare nucleic acids for downstream applications. The downstream applications may be utilized to, e.g., detect the presence or absence of a nucleic acid sequence present in the sample. In some instances, the devices and methods can be utilized to detect the presence or absence of one or more microbes in a biological sample. In some cases, the one or more microbes are pathogens (i.e., disease-causative). In some cases, the one or more microbes are infectious. In some cases, the one or more microbes cause disease in a subject. In some cases, the disease is a sexually transmitted disease.

In some aspects, the devices and methods can be utilized to detect the presence or absence of nucleic acids associated with one or more bacterial cells in the biological sample. In some cases, one or more bacterial cells are pathogens. In some cases, the one or more bacterial cells are infectious. Non-limiting examples of bacterial pathogens that can be detected include Mycobacteria (e.g. M. tuberculosis, M. bovis, M. avium, M. leprae, and M. africanum), rickettsia, mycoplasma, chlamydia, and legionella. Some examples of bacterial infections include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include, but are not limited to: Helicobacter pyloris, Legionella pneumophilia, Mycobacterium tuberculosis, Mycobacterium avium, Mycobacterium intracellulare, Mycobacterium kansaii, Mycobacterium gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Erysipelothrix rhusiopathiae, Clostridium tetani, Enterobacter aerogenes, Klebsiella pneumoniae, Pasteurella multocida, Fusobacterium nucleatum, Streptobacillus moniliformis, Treponema pallidium, Treponema pertenue, Leptospira, Rickettsia, and Actinomyces israelii, Acinetobacter, Bacillus, Bordetella, Borrelia, Brucella, Campylobacter, Chlamydia, Chlamydophila, Clostridium, Corynebacterium, Enterococcus, Haemophilus, Helicobacter, Mycobacterium, Mycoplasma, Stenotrophomonas, Treponema, Vibrio, Yersinia, Acinetobacter baumanii, Bordetella pertussis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Corynebacterium diphtheriae, Enterobacter sazakii, Enterobacter agglomerans, Enterobacter cloacae, Enterococcus faecalis, Enterococcus faecium, Escherichia coli, Francisella tularensis, Helicobacter pylori, Legionella pneumophila, Leptospira interrogans, Mycobacterium leprae, Mycobacterium tuberculosis, Mycobacterium ulcerans, Mycoplasma pneumoniae, Pseudomonas aeruginosa, Rickettsia rickettsii, Salmonella typhi, Salmonella typhimurium, Salmonella enterica, Shigella sonnei, Staphylococcus epidermidis, Staphylococcus saprophyticus, Stenotrophomonas maltophilia, Vibrio cholerae, Yersinia pestis, and the like. In some instances, the infectious bacteria is Neisseria gonorrhoeae or Chlamydia trachomatis.

In some aspects, the devices and methods can be utilized to detect the presence or absence of nucleic acids associated with one or more viruses in the biological sample. Non-limiting examples of types of viruses include double stranded DNA viruses, single stranded DNA viruses, double stranded RNA viruses, or single stranded RNA viruses. Single stranded RNA viruses may replicate directly or may include a DNA intermediate in their lifecycle. DNA viruses may replicate directly or through an RNA intermediate. Non-limiting examples of viruses include the herpes virus (e.g., human cytomegalomous virus (HCMV), herpes simplex virus 1 (HSV-1), herpes simplex virus 2 (HSV-2), varicella zoster virus (VZV), Epstein-Barr virus), influenza A virus and Hepatitis C virus (HCV) or a picornavirus such as Coxsackievirus B3 (CVB3). Other viruses may include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In some cases, the virus is an enveloped virus. Examples include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus. In some embodiments, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Birnavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses Al-22, 24 (CA1-22 and CA24, CA23 (echovirus 9)), human coxsackieviruses (Bl-6 (CBl-6)), human echoviruses 1-7, 9, 11-27, 29-33, vilyuish virus, simian enteroviruses 1-18 (SEV1-18), porcine enteroviruses 1-11 (PEV1-11), bovine enteroviruses 1-2 (BEV1-2), hepatitis A virus, rhinoviruses, hepatoviruses, cardio viruses, aphthoviruses and echoviruses. The virus may be phage. Examples of phages include, but are not limited to T4, T5, λ phage, T7 phage, G4, P1, φ6, Thermoproteus tenax virus 1, M13, MS2, Qβ, φX174, Φ29, PZA, Φ15, BS32, B103, M2Y (M2), Nf, GA-1, FWLBc1, FWLBc2, FWLLm3, B4. In some cases, the virus is selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus. In some cases, the virus is selected from a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus. In some cases, the virus is selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus. In some cases, the virus is selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxyiridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively).

In some aspects, the devices and methods can be utilized to detect the presence or absence of nucleic acids associated with one or more fungi in the biological sample. Examples of infectious fungal agents include, without limitation Aspergillus, Blastomyces, Coccidioides, Cryptococcus, Histoplasma, Paracoccidioides, Sporothrix, and at least three genera of Zygomycetes. The above fungi, as well as many other fungi, can cause disease in pets and companion animals. The present teaching is inclusive of substrates that contact animals directly or indirectly. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans. Further examples of fungal infectious agent include, but are not limited to, Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

In some aspects, the devices and methods can be utilized to detect the presence or absence of nucleic acids associated with one or more parasites in the biological sample. Non-limiting examples of parasites include Plasmodium, Leishmania, Babesia, Treponema, Borrelia, Trypanosoma, Toxoplasma gondii, Plasmodium falciparum, P. vivax, P. ovale, P. malariae, Trypanosoma spp., or Legionella spp. In some cases, the parasite is Trichomonas vaginalis.

In some cases, the biological sample can be an environmental sample comprising medium such as water, soil, air, and the like. In some cases, the biological sample can be a forensic sample (e.g., hair, blood, semen, saliva, etc.). In some cases, the biological sample can comprise an agent used in a bioterrorist attack (e.g., influenza, anthrax, smallpox).

In some aspects, the biological sample comprises an infectious agent associated with a sexually-transmitted disease (STD) or a sexually-transmitted infection (STI). Non-limiting examples of STDs or STIs and associated infectious agents that may be detected with the devices and methods provided herein may include, Bacterial Vaginosis; Chlamydia (Chlamydia trachomatis); Genital herpes (herpes virus); Gonorrhea (Neisseria gonorrhoeae); Hepatitis B (Hepatitis B virus); Hepatitis C (Hepatitis C virus); Genital Warts, Anal Warts, Cervical Cancer (Human Papillomavirus); Lymphogranuloma venereum (Chlamydia trachomatis); Syphilis (Treponema pallidum); Trichomoniasis (Trichomonas vaginalis); Yeast infection (Candida); and Acquired Immunodeficiency Syndrome (Human Immunodeficiency Virus).

Performance

In some cases, the devices and methods described herein may demonstrate improved performance when compared with traditional methods. For example, in some cases, the devices and methods may result in the extraction and preparation of nucleic acid molecules suitable for use in a polymerase chain reaction (PCR) in a shorter period of time when compared with other methods. In some cases, the devices and methods may result in the extraction and preparation of nucleic acid molecules suitable for use in a PCR reaction in 20 minutes or less. For example, the extraction and preparation of nucleic acid molecules as described herein may be achieved in about 20 minutes, 19 minutes, 18 minutes, 17 minutes, 16 minutes, 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes, 1 minute or less than 1 minute. In some cases, the extraction and preparation of nucleic acid molecules as described herein is achieved in about 5 minutes or less. In some cases, the method extracts nucleic acid molecules in about 5 minutes or less at a quality sufficient to successfully run a polymerase chain reaction (PCR).

A quality of extracted or prepared nucleic acid sufficient to run a polymerase chain reaction refers to the quantity of extracted or prepared nucleic acid, the purity of the nucleic acid and the shearing of the nucleic acid (average length of nucleic acid molecules). A sufficient quantity of nucleic acid may refer to about 0.001, 0.01, 0.05, 0.1, 0.2, 0.3, 0.4, 0.5, 0.6, 0.7, 0.8, 0.9, or 1 μg. A sufficient quantity may also refer to the concentration of the nucleic acid in the eluted liquid. The concentration of the eluted nucleic acid may be about 0.001, 0.01, 0.05, 0.1, 0.2, 0.3, 0.4, 0.5, 0.6, 0.7, 0.8, 0.9 or 1 μg/μL. The nucleic acid produced may comprise nucleic acid fragments with an average length of at least about 100, 200, 300, 400, 500, 600, 700, 800, 900, 1000 or more than 1000 base pairs.

A quality of extracted or prepared nucleic acid sufficient to run a polymerase chain reaction may be a sample that produces at least 70% efficiency as determined by a qPCR standard curve. The efficiency of the PCR may be between 90-100% (−3.6≥slope≥−3.3). Efficiency of qPCR may be quantified by calculating the cycle difference between a sample and 10-fold dilution of the sample. For example if the efficiency is 100%, the Ct values of a 10 fold dilution of input DNA will be 3.3 cycles apart (there is a 2-fold change for each change in Ct).

In some cases, the nucleic acid sample extracted or prepared using the devices and methods described herein have similar or improved purity as compared to nucleic acid samples prepared using other methods. The purity may be measured, for example, as a ratio of the absorbance at 260 nm and 280 nm (e.g., A260/A280). For example, a nucleic acid samples comprising DNA prepared using the devices and methods may have a A260/A280 ratio of about 1.5, about 1.6, about 1.7, about 1.8, about 1.9, or about 2.0. In some cases, the extracted or prepared nucleic acid molecules comprise DNA and the DNA has an A260/A280 ratio of at least 1.5. In another example, a nucleic acid sample comprising RNA prepared using the devices and methods may have an A260/A280 ratio of about 1.7, about 1.8, about 1.9, about 2.0, about 2.1, or about 2.2. In some cases, the extracted nucleic acid molecules comprise RNA and the RNA has an A260/A280 ratio of at least 1.7.

Downstream processes such as polymerase chain reaction (PCR) may be sensitive to certain molecules present in a sample. For example, the presence of one or more lysis reagents (e.g., Proteinase K) may hinder or inhibit downstream processes. In some cases, the nucleic acid molecules described herein are extracted from the one or more biological cells or entities with a quality that is sufficient to successfully perform one or more downstream processes. In some cases, the extracted nucleic acid molecules may be of a quality sufficient to successfully perform a PCR. For example, the extracted nucleic acid molecules may be of a quality sufficient to perform an amplification reaction on a target nucleic acid molecule present in the extracted nucleic acid molecules to generate amplified target nucleic molecules. In some cases, a positive control may be used (e.g., a biological cell that is known to be positive for the target molecule) to confirm that the extraction process is performed successfully. The extracted nucleic acid molecules described herein are generally substantially free of molecules that inhibit downstream processes (e.g., Proteinase K).

In some cases, the nucleic acid samples may have similar or improved yields as compared to nucleic acid samples prepared using other methods from the same amount of starting material. For example, nucleic acid samples prepared using the methods and devices described herein may have about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or greater yields than using other nucleic acid extraction methods from the same amount of starting material.

Standard nucleic acid extraction methods may involve the use of centrifuges and vacuums. In some cases, the methods and devices herein do not involve the use of centrifuges or vacuums.

Devices

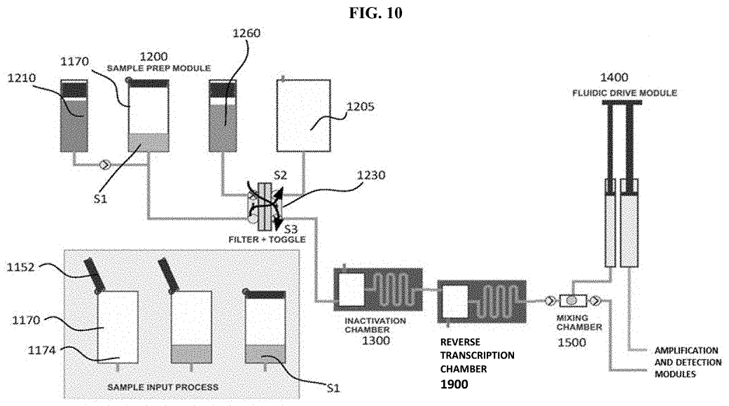

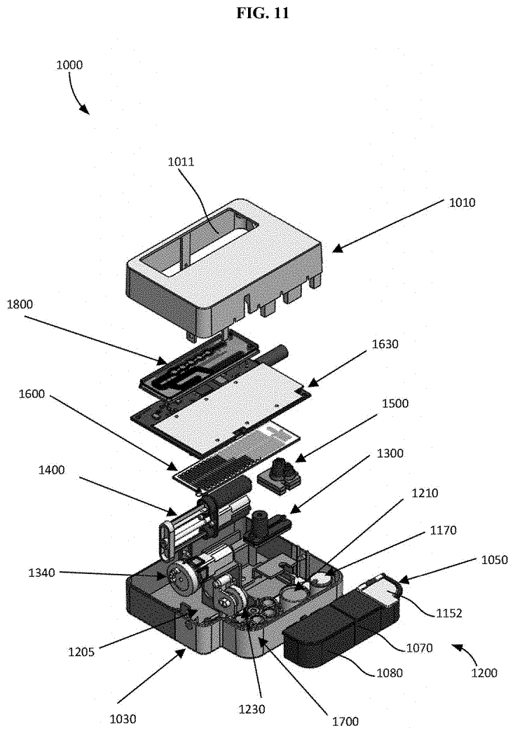

In some aspects, devices are provided for performing any of the methods described herein. For example, FIG. 10 is a schematic illustration of a molecular diagnostic test device 1000 (also referred to as a “test device” or “device”), according to an embodiment. The schematic illustration describes the primary components of the test device 1000 as shown in FIG. 11. The test device 1000 is an integrated device (i.e., the modules are contained within a single housing) that is suitable for use within a point-of-care setting (e.g., doctor's office, pharmacy or the like), decentralized test facility, or at the user's home. In some embodiments, the device 1000 can have a size, shape and/or weight such that the device 1000 can be carried, held, used and/or manipulated in a user's hands (i.e., it can be a “handheld” device). A handheld device may have dimensions less than 15 cm×15 cm×15 cm, or less than 15 cm×15 cm×10 cm, or less than 12 cm×12 cm×6 cm. In other embodiments, the test device 1000 can be a self-contained, single-use device. In some embodiments, the test device 1000 can be configured with lock-outs or other mechanisms to prevent re-use or attempts to re-use the device.

Further, in some embodiments, the device 1000 can be a CLIA-waived device and/or can operate in accordance with methods that are CLIA waived. Similarly stated, in some embodiments, the device 1000 (and any of the other devices shown and described herein) is configured to be operated in a sufficiently simple manner, and can produce results with sufficient accuracy to pose a limited likelihood of misuse and/or to pose a limited risk of harm if used improperly. In some embodiments, the device 1000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 1000 can be configured for long term storage in a manner that poses a limited likelihood of misuse (spoilage of the reagent(s), expiration of the reagents(s), leakage of the reagent(s), or the like). In some embodiments, the molecular diagnostic test device 1000 is configured to be stored for up to about 36 months, up to about 32 months, up to about 26 months, up to about 24 months, up to about 20 months, up to about 18 months, or any values there between.

The test device 1000 is configured to manipulate a biological sample S1 to produce one or more output signals associated with a target cell. Specifically, the device 1000 includes a sample preparation module 1200, an inactivation module 1300 (also referred to as a lysing module), a fluidic drive (or fluid transfer) module 1400, a mixing chamber 1500, an amplification module, a detection module and a power and control module (not shown). The test device and certain components therein can be similar to any of the molecular test devices shown and described herein or in International Patent Publication No. WO2016/109691, entitled “Devices and Methods for Molecular Diagnostic Testing,” which is incorporated herein by reference in its entirety. Accordingly, a detailed description of certain modules (e.g., the fluidic drive module 1400) is not provided herein. A description of each of the modules is provided below.

FIG. 11 shows a perspective exploded view of the molecular diagnostic test device 1000. The diagnostic test device 1000 includes a housing (including a top portion 1010 and a bottom portion 1030), within which the modules described herein are contained. Similarly stated, the housing (including the top portion 1010 and/or the bottom portion 1030) surround and/or enclose the modules. As shown, the top housing 1010 defines a detection opening 1011 that is aligned with the detection module 1800 such that the signal produced by and/or on each detection surface of the detection module 1800 is visible through the detection opening 1011. In some embodiments, the top housing 1010 and/or the portion of the top housing 1010 surrounding the detection opening 1011 is opaque (or semi-opaque), thereby “framing” or accentuating the detection openings. In some embodiments, for example, the top housing 1010 can include markings (e.g., thick lines, colors or the like) to highlight the detection opening 1011. For example, in some embodiments, the top housing 1010 can include indicia identifying the detection opening to a specific disease (e.g., Chlamydia trachomatis (CT), Neisseria gonorrhea (NG) and Trichomonas vaginalis (TV)) or control. In other embodiments, the top housing 1010 can include a series of color spots having a range of colors associated with a range of colors that is likely produced by the signals produced during the test. In this manner, the housing design can contribute to reducing the amount of user judgment required to accurately read the test.

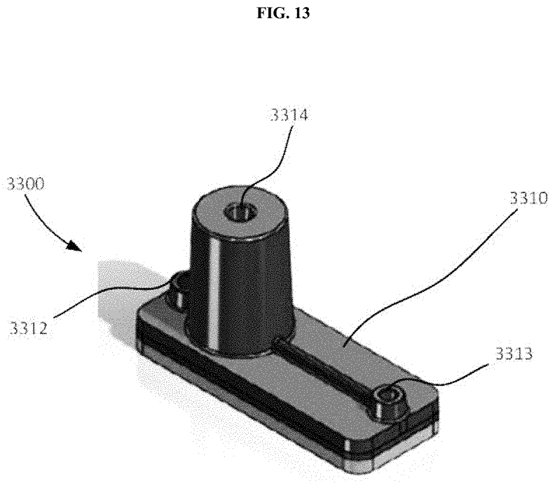

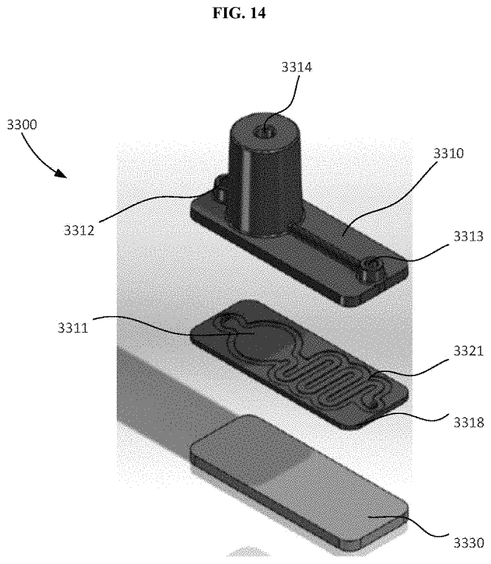

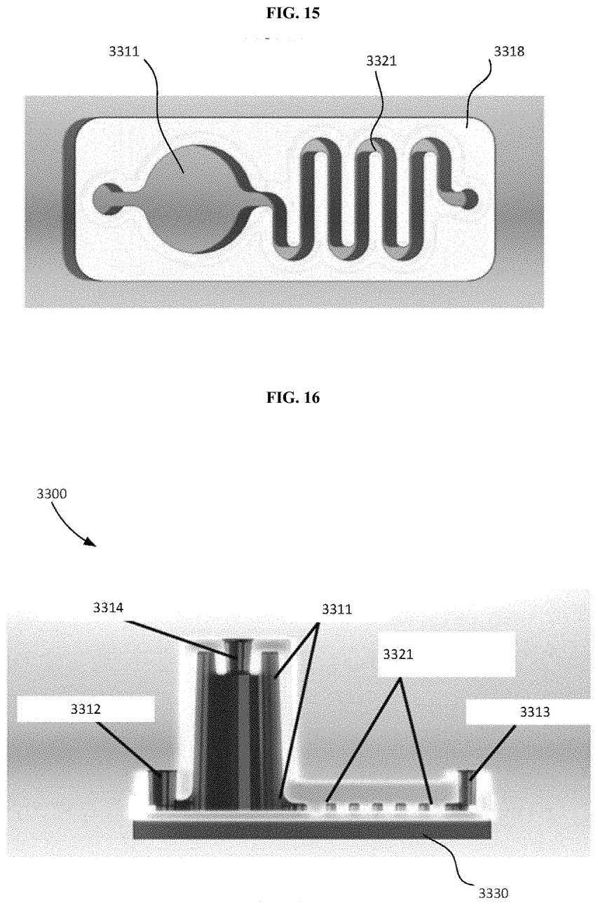

Referring to FIG. 11, the sample preparation module 1200 includes a sample input module 1170, a wash module 1210, an elution module 1260, a filter assembly 1230, and various fluidic conduits (e.g., tubes, lines, valves, etc.) connecting the various components. The device 1000 also includes the lysing module 1300 (see e.g., the lysing module 2300 shown in FIGS. 13-16), which, together with the sample preparation module 1200, performs the nucleic acid extraction according to any of the methods described herein. Thus, although the sample preparation module 1200 and the inactivation module 1300 are described as two separate modules, in other embodiments, the structure and function of the sample preparation module 1200 can be included within or performed by the inactivation module 1300 and vice-versa. Similarly stated, any of the sample preparation modules, inactivation modules and/or lysing modules described herein can include any of the structure and/or perform any of the functions of the other modules to perform any of the methods of sample preparation or nucleic acid extraction described herein. By eliminating the need for external sample preparation and a cumbersome instrument, the device 1000 is suitable for use within a point-of-care setting (e.g., doctor's office, pharmacy or the like) or at the user's home, and can receive any suitable biological sample S1. The biological sample S1 (and any of the input samples described herein) can be, for example, blood, urine, male urethral specimens, vaginal specimens, cervical swab specimens, and/or nasal swab specimens gathered using a commercially available sample collection kit.

The sample input module 1170 is disposed within the housing 1010, and is configured receive a biological sample S1 containing a biological entity. The biological sample S1 can be any of the sample types described herein, and the biological entity can be any of the entities described herein. The sample input module 1170 defines a sample volume 1174 that can be selectively covered by the cap 1152. The cap 1152 can include seals or other locking members such that it can be securely fastened to the lower housing 1030 (or other portions of the device 1000) and/or can be closed during shipping, after delivery of a sample thereto, or the like. In some embodiments, the input port cap 1152 can include an irreversible lock to prevent reuse of the device 1000 and/or the addition of supplemental sample fluids. In this manner, the device 1000 can be suitably used by untrained individuals.

The wash module 1210 includes a housing that defines a wash volume containing any suitable wash composition. For example, in some embodiments, the wash module 1210 can include a gaseous first wash composition (e.g., nitrogen, air, or another inert gas) and a liquid second wash composition. In this manner, the wash operation can include an “air purge” of the filter assembly 1230. Specifically, when the sample input module 1170 and/or the wash module 1210 is actuated, a serial flow of the first wash composition (gas) followed by the second wash composition (liquid). By first including a gas (or air) wash (i.e., the first wash composition), the amount of liquid constituents from the input sample conveyed to the filter assembly 1230 (indicated by the flow S2 in FIG. 10) can be reduced. Said another way, after delivery of the input sample, the filter assembly 1230 will retain the desired sample cells (or organisms) and some amount of residual liquid. By forcing the first, gaseous wash composition through the filter (i.e., an “air wash”), the amount of residual liquid can be minimized. This arrangement can reduce the amount of liquid wash (e.g., the second wash composition) needed to sufficiently prepare the sample particles. Reducing the liquid volume contributes to the reduction size of the device 1000, and also reduces the likelihood of potentially harmful shearing stress when the liquid wash is flowed through the filter assembly 1230.

The sample input module 1170 (and any of the sample input modules described herein) and the wash module 1210 (and any of the wash modules described herein) can be actuated by any suitable mechanism to convey the biological sample S1 towards the filter assembly 1230 and/or the lysing module 1300 to enable the nucleic acid extraction methods described herein. For example, in the embodiment shown, the sample input module 1170 and the wash module 1210 are actuated by the sample actuator (or button) 1050. The sample actuator 1050 is movably coupled to the housing, and is aligned with and can move a piston or plunger (not shown) within the sample volume 1174 when the sample input module 1170 is actuated. Thus, the sample actuator 1050 is a non-electronic actuator that is manually depressed by a user to actuate the sample input module 1170. In other embodiments, however, the sample actuator 1050 can be an electronic actuator. In some embodiments, the sample actuator 1050 can include a lock tab (not shown) that is fixedly received within the notch or opening of the housing 1010 to fix the sample actuator 1050 in its second or “actuated” position, as described above. In this manner, the device 1000 cannot be reused after the initial actuation.

When actuated, the sample within the sample volume 1174 is conveyed along with the wash solution(s) from the wash module 1210 towards the filter assembly 1230. The flow of the biological sample S1 towards the filter assembly 1230 is shown by the arrow S2 in FIG. 10. The filter assembly 1230 is configured to filter and prepare the biological sample S1 (via the sample input operation and the sample wash operation), and to allow a back-flow elution operation to deliver captured particles from the filter membrane and deliver the eluted volume to lysing module 1300. The filter assembly 1230 can be toggled between two configurations to allow the flow of the biological sample S1 and wash solution in a first direction (towards the waste reservoir 1205), followed by a backflush of the elution reagent and the captured organisms (or cells) in a second direction (as indicated by the arrow S3 towards the lysing/inactivation module 1300). The toggling mechanism can be any suitable mechanism, such as those shown and described in International Patent Publication No. WO2016/109691, entitled “Devices and Methods for Molecular Diagnostic Testing,” which is incorporated herein by reference in its entirety.

The filter assembly 1230 can include any suitable filter membrane that captures the target organism/entity while allowing the bulk of the liquid within the biological sample S1, the first wash composition, and the second wash composition to flow therethrough and into the waste tank 1205. The filter membrane 1254 (and any of the filter membranes described herein) can be any suitable membrane and or combination of membranes as described herein. For example, in some embodiments, the filter membrane 1254 is a woven nylon filter membrane with a pore size of about 1 μm (e.g., 0.8 μm, 1.0 μm, 1.2 μm) enclosed between various plates of the filter assembly 1230 such that there is minimal dead volume.

The elution module (or assembly) 1260 of the sample preparation module 1200 is contained within the housing, and defines an elution volume within which an elution composition is stored. The elution composition can be any of the elution compositions described herein. In some embodiments, the elution composition can include proteinase K, which allows for the release of any bound cells and/or nucleic acid molecules (e.g., DNA) from the filter membrane. The output from the elution module 1260 can be selectively placed in fluid communication with the filter assembly 1230, when the filter assembly is toggled into its second (or backflow) configuration. Thus, the elution module 1230 can include any suitable flow control devices, such as check valves, duck-bill valves, or the like to prevent flow back towards and/or into the elution volume.

The elution module 1210 is actuated by the elution actuator (or button) 1070 (see FIG. 11). The reagent actuator 1070 is movably coupled to the lower housing 1030, and can exert force on a piston or other portion of the elution module 1210 to convey the elution composition back through the filter and towards the lysing module 1300, as shown by the arrow S3. In some embodiments, the elution actuator 1070 further includes a lock tab or other structure that is fixedly received within the notch or opening of the housing to fix the elution actuator 1070 in its second or “actuated” position. In this manner, the device 1000 cannot be reused after the actuation of the elution actuator.

In use, the filter assembly 1230 recovers the target organisms with a certain efficiency, from a given starting volume. The wash operation then removes undesired material, without removing the target organisms (which stay present on the filter membrane). The elution operation then removes the target organism from the filter membrane, diluting the total amount of captured organisms in the volume of the elution solution, thus comprising the eluent. By modifying the total output volume of eluent, a higher or lower concentration of both target organism and any potential inhibiting matter can be achieved. In some embodiments, a further dilution can be achieved, if desired, by mixing the eluent solution with another reagent after the initial sample preparation. Given a known volume of eluent, and a known volume of diluent, a correct dilution factor can be achieved, through to maintain the reliability of the system very high dilution factors are avoided.

As shown by the arrow S3 in FIG. 10, the elution solution and the captured cells and/or organisms are conveyed during the elution operation back through the filter assembly 1230, and to the inactivation module (or lysing module) 1300. In some examples, such as that shown by arrow S3 in FIG. 10, the elution step may involve the nucleic acids, cells, or biological entities passing through the filter. In other examples the elution step may involve washing the nucleic acids, cells, or biological entities off the filter, such that they remain on the same side of the filter without passing through it. The inactivation module 1300 is configured to be fluidically coupled to and receive the eluted sample S3 from the sample preparation module 1200. In some embodiments, the inactivation module 1300 is configured for lysis of the received input fluid. In some embodiments, the inactivation module 1300 is configured for de-activating the enzymes present in input fluid after lysis occurs. In some embodiments, the inactivation module 1300 is configured for preventing cross-contamination between the output fluid and the input fluid. The inactivation module 1300 can include any of the inactivation (or lysing) modules as described herein, including the lysing module 3300 and the lysing module 4300 described herein.

In some embodiments, the sample is transferred from the inactivation module to a reverse transcription module 1900. In some embodiments, the reverse transcription module is configured to incubate the sample at a temperature suitable for a reverse transcription enzyme, and subsequently incubate the sample at a temperature high enough to deactivate the reverse transcriptase enzyme. The reverse transcription module 1900 may include any of the reverse transcription modules described herein.

In some embodiments the reverse transcription module 1900 is omitted from the device and a reverse transcriptase enzyme is present in the amplification module or the mixing module. In this embodiment the reverse transcriptase enzyme is chosen to be one which is active under the conditions required for the amplification reaction. Alternatively the DNA polymerase enzyme may be chosen for activity under the conditions required by the reverse transcriptase enzyme. The amplification module is capable of heating the solution to the temperatures required for reverse transcription and inactivation of the reverse transcriptase enzyme, as well as the temperatures required by the DNA polymerase enzyme.

The mixing module (also referred to as simply the mixing chamber) 1500 mixes the output of inactivation module 1300 with the reagents to conduct a successful amplification reaction. Similarly stated, the mixing module 1500 is configured to reconstitute the reagent in a predetermined input volume, while ensuring even local concentrations of reagents in the entirety of the volume. In some embodiments, the mixing chamber module 1500 is configured to produce and/or convey a sufficient volume of liquid for the amplification module 1600 to provide sufficient volume output to the detection module 1800. The mixing module 1500 can be any suitable mixing module, such as those shown and described in International Patent Publication No. WO2016/109691, entitled “Devices and Methods for Molecular Diagnostic Testing,” which is incorporated herein by reference in its entirety.

The fluidic drive (or transfer) module 1400 can be a pump or series of pumps configured to produce a pressure differential and/or flow of the solutions within the diagnostic test device 1000. Similarly stated, the fluid transfer module 1400 is configured to generate fluid pressure, fluid flow and/or otherwise convey the biological sample S1, and the reagents through the various modules of the device 1000. The fluid transfer module 1400 is configured to contact and/or receive the sample flow therein. Thus, in some embodiments, the device 1000 is specifically configured for a single-use to eliminate the likelihood that contamination of the fluid transfer module 1400 and/or the sample preparation module 1200 will become contaminated from previous runs, thereby negatively impacting the accuracy of the results. The fluid transfer module 1500 can be any suitable fluid transfer module, such as those shown and described in International Patent Publication No. WO2016/109691, entitled “Devices and Methods for Molecular Diagnostic Testing,” which is incorporated herein by reference in its entirety.

After being mixed within the mixing module 1500, the prepared sample is then conveyed to the amplification module 1600 (as shown by the arrow CC in FIG. 10). The amplification module 1600 includes a flow member 1610 and a heater 1630. The flow member 1610 can be any suitable flow member that defines a volume or a series of volumes within which the that prepared solution S3 can flow and/or be maintained to amplify the target nucleic acid molecules within the solution S3. The heater 1630 can be any suitable heater or group of heaters coupled to the flow member 1610 that can heat the prepared solution within the flow member 1610 to perform any of the amplification operations as described herein. For example, in some embodiments, the amplification module 1600 (or any of the amplification modules described herein) can be similar to the amplification modules shown and described in U.S. patent application Ser. No. 15/494,145, entitled “Printed Circuit Board Heater for an Amplification Module,” which is incorporated herein by reference in its entirety. In other embodiments, the amplification module 1600 (or any of the amplification modules described herein) can be similar to the amplification modules shown and described in International Patent Publication No. WO2016/109691, entitled “Devices and Methods for Molecular Diagnostic Testing,” which is incorporated herein by reference in its entirety.

In some embodiments, the flow member 1610 defines a single volume within which the prepared solution is maintained and heated to amplify the nucleic acid molecules within the prepared solution. In other embodiments, the flow member 1610 can define a “switchback” or serpentine flow path through which the prepared solution flows. Similarly stated, the flow member 1610 defines a flow path that is curved such that the flow path intersects the heater 1630 at multiple locations. In this manner, the amplification module 1600 can perform a “flow through” amplification reaction where the prepared solution flows through multiple different temperature regions.

The flow member 1610 (and any of the flow members described herein) can be constructed from any suitable material and can have any suitable dimensions to facilitate the desired amplification performance for the desired volume of sample. For example, in some embodiments, the amplification module 1600 (and any of the amplification modules described herein) can perform 1000λ or greater amplification in a time of less than 15 minutes. For example, in some embodiments, the flow member 1610 (and any of the flow members described herein) is constructed from at least one of a cyclic olefin copolymer or a graphite-based material. Such materials facilitate the desired heat transfer properties into the flow path. Moreover, in some embodiments, the flow member 1610 (and any of the flow members described herein) can have a thickness of less than about 0.5 mm. In some embodiments, the flow member 1610 (and any of the flow members described herein) can have a volume about 150 microliters or greater, and the flow can be such that at least 10 microliters of sample is amplified. In other embodiments, at least 20 microliters of sample are amplified by the methods and devices described herein. In other embodiments, at least 30 microliters of sample are amplified by the methods and devices described herein. In yet other embodiments, at least 50 microliters of sample are amplified by the methods and devices described herein.

The heater 1630 can be any suitable heater or collection of heaters that can perform the functions described herein to amplify the prepared solution. In some embodiments, the heater 1630 can establish multiple temperature zones through which the prepared solution flows and/or can define a desired number of amplification cycles to ensure the desired test sensitivity (e.g., at least 30 cycles, at least 34 cycles, at least 36 cycles, at least 38 cycles, or at least 40 cycles). The heater 1630 (and any of the heaters described herein) can be of any suitable design. For example, in some embodiments, the heater 1630 can be a resistance heater, a thermoelectric device (e.g. a Peltier device), or the like. In some embodiments, the heater 1630 can be one or more linear “strip heaters” arranged such that the flow path crosses the heaters at multiple different points. In other embodiments, the heater 1630 can be one or more curved heaters having a geometry that corresponds to that of the flow member 1610 to produce multiple different temperature zones in the flow path.

Although the amplification module 1600 is generally described as performing a thermal cycling operation on the prepared solution, in other embodiment, the amplification module 1600 can perform any suitable thermal reaction to amplify nucleic acids within the solution. In some embodiments, the amplification module 1600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process

The detection methods enabled by the device 1000 include sequential delivery of the detection reagents and other substances within the device 1000. Further, the device 1000 is configured to be an “off-the-shelf” product for use in a point-of-care location (or other decentralized location), and is thus configured for long-term storage. Accordingly, the reagent storage module 1700 is configured for simple, non-empirical steps for the user to remove the reagents from their long-term storage containers, and for removing all the reagents from their storage containers using a single user action. In some embodiments, the reagent storage module 1700 and the rotary selection valve 1340 are configured for allowing the reagents to be used in the detection module 1800, one at a time, without user intervention.

Specifically, the device 1000 is configured such that the last step of the initial user operation (i.e., the depressing of the reagent actuator 1080) results in dispensing the stored reagents. This action crushes and/or opens the sealed reagent containers present in the assembly and relocates the liquid for delivery. The rotary venting selector valve 1340 allows the reagent module 1700 to be vented for this step, and thus allows for opening of the reagent containers, but closes the vents to the tanks once this process is concluded. Thus, the reagents remain in the reagent module 1700 until needed in the detection module 1800. When a desired reagent is needed, the rotary valve 1340 opens the appropriate vent path to the reagent module 1700, and the fluidic drive module 1400 applies vacuum to the output port of the reagent module 1700 (via the detection module 1800), thus conveying the reagents from the reagent module 1700. The reagent module 1700 and the valve 1340 can be similar to the reagent modules and valves shown and described in International Patent Publication No. WO2016/109691, entitled “Devices and Methods for Molecular Diagnostic Testing,” which is incorporated herein by reference in its entirety.

The detection module 1800 is configured to receive output from the amplification module 1600 and reagents from the reagent module 1700 to produce a colorimetric change to indicate presence or absence of target organism in the initial input sample. The detection module 1800 also produces a colorimetric signal to indicate the general correct operation of the test (positive control and negative control). In some embodiments, color change induced by the reaction is easy to read and binary, with no requirement to interpret shade or hue. The detection module 1800 can be similar to the detection modules shown and described in International Patent Publication No. WO2016/109691, entitled “Devices and Methods for Molecular Diagnostic Testing,” which is incorporated herein by reference in its entirety.

In one aspect, a device is provided comprising: (a) an input port, configured to receive the biological sample comprising one or more biological cells or biological entities; (b) a filter assembly comprising a filter configured to capture the one or more biological cells or biological entities, wherein the input port is configured to relay the biological sample to the filter assembly; (c) one or more reservoirs comprising a wash solution, a lysis solution, or both, operably coupled to the filter assembly; (d) a waste chamber, operably coupled to the filter assembly and configured to receive waste from the filter assembly; and (e) an elution chamber, operably coupled to the filter assembly and configured to receive an eluent from the filter assembly.

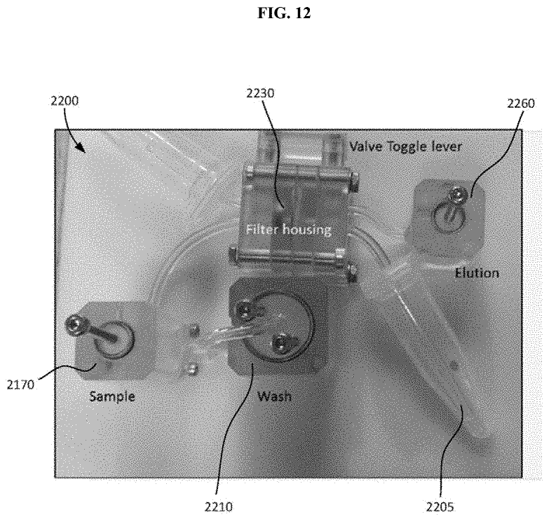

For example, FIG. 12 depicts an example of a sample preparation device (or module) 2200 that may be used to perform the methods provided herein. The sample preparation module 2200 can be included in any of the molecular diagnostic test devices described herein, including the device 1000 described above. It should be understood that the invention is not limited to a particular arrangement or configuration of the sample preparation device, and any suitable arrangement or configuration may be used. In some cases, the sample preparation device 2200 comprises an input port 2170. The input port is configured to receive a sample (e.g., biological sample). For example, the input port 2170 may be configured to receive about 50 μL to about 20 mL of a liquid sample. The input port 2170 may comprise a reservoir or chamber for holding or storing the sample. The input port 2170 may comprise a cap or lid (similar to the lid 1152 described above) that can be placed over the input port to contain the sample in the reservoir or chamber. The input port 2170 may be operably coupled to a filter assembly 2230. In use, the sample may be relayed (e.g., pushed or flowed) to the filter assembly 2230 in any manner as described herein. The filter assembly 2230 may contain one or more filter membranes for capturing biological cells or entities on the filter. In some instances, the filter assembly 2230 (or any of the filter assemblies described herein) contains at least two filter membranes, one with a larger pore size and one with a smaller pore size. The two filter membranes may be arranged such that the sample first passes through the membrane with the larger pore size and then the membrane with the smaller pore size. The filter membrane may be of any suitable material as described herein, non-limiting examples including nylon, cellulose, polyethersulfone (PES), polyvinylidene difluoride (PVDF), polycarbonate, borosilicate glass fiber and the like. In some examples, the filter membrane is nylon. In some cases, the filter membrane has an average pore size of about 0.2 μm to about 20 μm. For example, the filter membrane may have an average pore size of about 0.2 μm, about 0.5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, or greater than 20 μm. In some examples, the surface of the filter membrane may be chemically treated or coated in such a way as to improve the binding of a biological cell or entity to the membrane. The biological cells or entities may be captured on the membrane while the majority of the liquid (“flow-through”) is flowed through the filter membrane. In some cases, the flow-through is substantially devoid of biological cells or entities. In some cases, the flow-through is disposed of by relaying the flow-through to one or more waste chambers operably coupled to the filter assembly. In other cases, the flow-through is relayed to a collection chamber for further downstream processing.

In some aspects, the sample preparation device 2200 further comprises one or more chambers 2210 or reservoirs for housing a wash solution. The one or more chambers or reservoirs (also referred to as wash modules) housing the wash solution may be operably coupled to the filter assembly such that actuation of the wash chamber or reservoir 2210 relays the wash solution to the filter assembly 2230. In some cases, the wash solution is provided as a lyophilized pellet or bead that sits within the chamber or reservoir. The lyophilized pellet or bead can be reconstituted in one or more solutions. The wash solution may be flowed through the filter assembly 2230 and the majority of the liquid can be collected in the one or more waste chambers 2205. Non-limiting examples of wash solutions suitable for use with the sample preparation device have been described above.

In certain aspects, the sample preparation device further comprises one or more chambers or reservoirs for housing a lysis solution. The chamber or reservoir housing the lysis solution may be operably coupled to the filter assembly such that actuation of the chamber or reservoir relays the lysis solution to the filter assembly. In some cases, the lysis solution may be flowed through the filter assembly. The lysis solution may cause the lysis or disruption of the biological cells or entities on the filter membrane. In some cases, the reagents of the lysis solution are provided as a lyophilized pellet or bead that sits within the chamber or reservoir (e.g., within a lysing module, similar to the lysing modules 1300, 3300 and 4300 described herein). The lyophilized pellet or bead can be reconstituted in one or more solutions. In some cases, the lysis enzyme is stored separately as a lyophilized bead or pellet within the device. In some cases, the lyophilized lysis enzyme may be reconstituted in the lysis buffer prior to addition to the cells. In other cases, the cells are eluted from the filter membrane and relayed into the elution chamber 2260 which contains the lyophilized lysis enzyme, thereby reconstituting the enzyme. In cases where a lysis enzyme is used, the enzyme is stable in the device at ambient temperatures for long periods of time. For example, the enzyme may be stable in the device at ambient temperature for at least one day, at least two days, at least three days, at least four days, at least five days, at least six days, at least one week, at least two weeks, at least three weeks, at least four weeks, at least a month, at least two months, at least three months, at least four months, at least five months, at least six months, at least seven months, at least eight months, at least nine months, at least ten months, at least eleven months, at least one year, at least two years, at least three years, at least four years, at least five years, at least six years, at least seven years, at least eight years, at least nine years, at least ten years or longer. The lysis solution containing the lysed cells (“eluent”) may be collected in an elution chamber. In some cases, the lysis solution may be back-flowed through the filter assembly. In this instance, the biological cells or entities on the filter membrane may be pushed or washed from the membrane and collected in an elution chamber with the lysis solution. The cells or entities (or lysed or otherwise disrupted cells or entities) diluted in the lysis solution may be referred to as the “eluent.”

In some aspects, the sample preparation device 2200 may further comprise one or more heating modules (not shown). The one or more heating modules may be operably coupled to the elution chamber 2260. The one or more heating modules may heat the elution chamber to a temperature sufficient for lysis of the biological cells or entities to occur. In some cases, the lysis solution comprises one or more enzymes (e.g., Proteinase K). In some cases, the one or more heating modules heats the elution chamber to a temperature sufficient for optimal performance of the lysis enzyme. In some examples, the heating module heats the elution chamber (and the fluid contained therein) to a temperature of about 4° C., about 10° C., about 15° C., about 20° C., about 25° C., about 30° C., about 40° C., about 45° C., about 50° C., about 55° C., about 60° C., about 65° C., about 70° C., about 75° C. or greater than 75° C.

In some aspects, the sample preparation device 2200 and/or any of the molecular diagnostic devices described herein further comprises an inactivation chamber (also referred to as an inactivation module or a lysing module). The inactivation chamber may be operably coupled to the elution chamber. The eluent may be relayed from the elution chamber to the inactivation chamber. In some instances, the elution chamber and the inactivation chamber are the same chamber and are coupled to a heating element that can heat the chamber to an optimal lysis temperature, and can further heat the chamber to an optimal inactivation temperature (e.g., from about 56° C. to about 95° C.).

For example, a non-limiting example of an inactivation chamber 3300 is depicted in FIGS. 13-16. In this example, the inactivation chamber comprises a chamber body 3310, a bottom lid 3318, and a heater 3330. As depicted in FIG. 12, the chamber body 3310 may defines an input port 3312, a holding tank (or first volume) 3311, a permanent vent 3314, an inactivation segment (or second volume) 3321, and an output port 3313. The input port 3312 may be configured to receive the eluent from the elution chamber and/or directly from a filter assembly (e.g., the filter assembly 1230). In other embodiments, as described herein, the input port 3312 can be fluidically coupled to a sample input module without the biological input being conveyed through a filter. The eluent may flow into the inactivation chamber (or lysing module 3300) and be collected in the holding tank 3311. The holding tank may have a capacity of about 1 μL to about 100 mL, about 100 μL to about 10 mL, about 300 μL to 1 mL, or about 300 μL to about 650 μL. The holding tank may be used to lyse the sample. For example, in some embodiments, the eluent containing the target organisms can be heated by the heater 3330 to maintain the eluent at or above a target lysing temperature. Similarly stated, in some embodiments, the heater 3330 can be coupled to the chamber body 3310 and/or the bottom lid 3318 such that the heater 3330 can convey thermal energy into the lysing module 3300 to produce a lysing temperature zone within the holding tank (or first volume) 3311. The lysing temperature zone can maintain the eluent at any of the temperatures and for any of the time periods described herein.

The vent 3314 may be a hole which allows air to flow into or out of the lysing module 3300 (including the first volume 3311 and the second volume 3321) as sample is brought in or out. The vent 3314 can also relieve pressure within either of the first volume 3311 or the second volume 3321 when the eluent is heated. Although described as being a permanent vent (i.e., a vent having a fixed opening), in some embodiments, the lysing module 3300 (or any of the lysing modules described herein) can have an active vent. For example, in some embodiments, the lysing module 3300 (or any of the lysing modules described herein) can include a valve that controls the venting of pressure and/or air from within the lysing module 3300.

The eluent may flow from the holding tank 3311 through the inactivation segment of the lysing module 3300. More specifically, the holding tank 3311 is in fluid communication with the inactivation segment 3321 such that when a pressure gradient is applied across the input port 3312 and the output port 3313, the eluent can flow from the holding tank 3311 (first volume) through the inactivation segment 3321 (second volume). The pressure gradient can be applied by any suitable mechanism, such as for example, a pump (e.g., the fluidic drive module 1400). The inactivation segment 3321 may be a small, shallow channel that allows efficient and rapid heating of the eluent as it leaves the holding tank. In a non-limiting example, the inactivation segment 3321 is configured in a serpentine pattern. The serpentine pattern may allow for rapid inactivation of the lysis enzymes in the eluent. The eluent, after being flowed through the inactivation segment, may be flowed into the output port 3313 to be collected. The volume of liquid passed through the heated channel could be from about 1 μL to about 100 mL, about 10 μL to about 10 mL, about 100 to about 5 mL, or about 250 μL to about 750 μL.

As described above, the inactivation module 3300 may be in contact with a heating element 3330, which can be, for example, a printed circuit board (PCB) heater. The heating element 3330 may function to heat the eluent as it flows through the inactivation segment at a high temperature sufficient to inactivate the one or more lysis enzymes contained within the eluent. For example, the heating element may heat the eluent to about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C. By heating the liquid eluent to a high temperature, the lysis enzymes as well as any other enzymes present can be deactivated. In some embodiments, the sample can be heated to about 95 C for about 3 minutes. In some embodiments, the serpentine path 3321 may be preceded by a check valve (not shown) to maintain a back pressure such that fluid does not enter the serpentine path 3321 before the desired temperature has been achieved. The serpentine area may be preheated to the desired temperature (50° C. to 99° C. or more) before fluid is drawn through the serpentine channel. If fluid were to flow into the serpentine channel prematurely without controlled flow, large bubbles may form in the channel as the heater warms up which could result in portions of the fluid to pass through the channel without receiving the proper temperature treatment.

In some embodiments there may be a one-way check valve that allows flow between the inactivation chamber and the mixing chamber (and prevents reverse flow). However, before flow can occur a certain amount of “cracking pressure” must be achieved. If the holding tank of the inactivation chamber is well vented from a vent port, the liquid that is placed into the holding tank will not flow into the serpentine channel due to the cracking pressure of the check valve at the exit of the serpentine channel. The cracking pressure may be from 0.05 to 50 psi. In some examples, the check valves used may have a cracking pressure of approximately 0.5 psi.

As described, the solution within the second volume 3321 is rapidly heated to temperatures of up to about 100 degrees Celsius. The lysing module 3300 and/or the formulation of the input solution (e.g., the eluent), however, can collectively reduce the likelihood that the liquid portion of the input solution will boil during the lysing/inactivation operations. Such boiling can produce undesirable bubbles and/or air pockets and can reduce the repeatability of the lysing and/or inactivation operations. Moreover, to facilitate use of the device at a variety of different altitudes, the lysing module 3300 and/or the formulation of the input solution can collectively reduce the likelihood that the liquid portion of the input solution will boil at a temperature of 99 degrees Celsius or higher, 98 degrees Celsius or higher, 96 degrees Celsius or higher, 94 degrees Celsius or higher, 92 degrees Celsius or higher, 90 degrees Celsius or higher, or 88 degrees Celsius or higher. For example, in some embodiments, the input solution can include salts and/or sugars to raise the boiling temperature of the input solution. In other embodiments, the lysing module 3300 can include one or more vent openings into either the first volume 3311 or the second volume 3321 or both (to limit pressure build-up during heating).

After the lysing and inactivation operations, the output from the lysing module 3300 can be conveyed into an (e.g., the amplification module 1600 or any other amplification modules described herein). Similarly stated, the output from the lysing module 3300, which contains the extracted nucleic acid molecules, can be conveyed to an amplification module. The amplification module can then perform a thermal reaction (e.g., an amplification reaction) on the prepared solution containing target nucleic acid mixed with required reagents. In some embodiments, the amplification module is configured to conduct rapid amplification of an input target. In some embodiments, the amplification module is configured to generate an output copy number that reaches or exceeds the threshold of the sensitivity of an associated detection module (e.g., the detection module 1800).

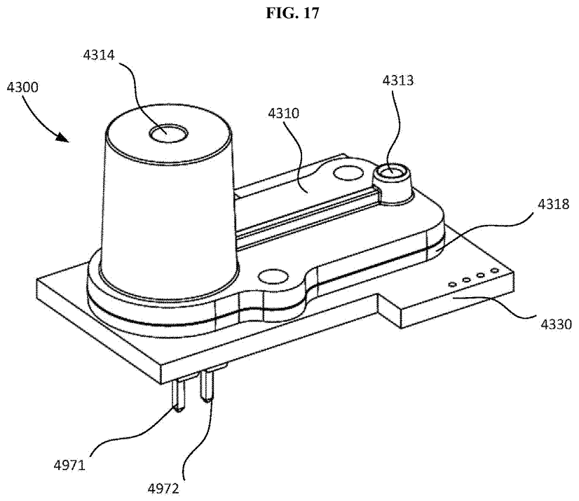

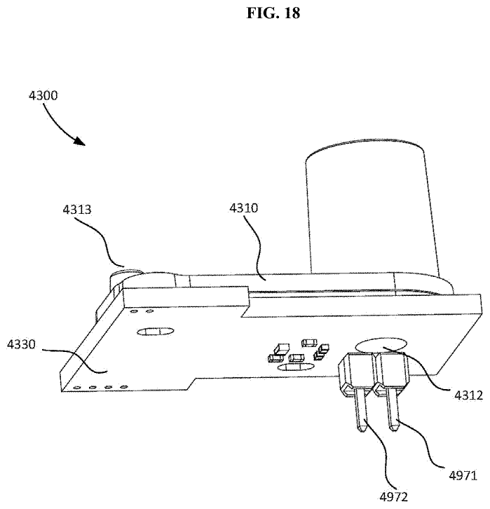

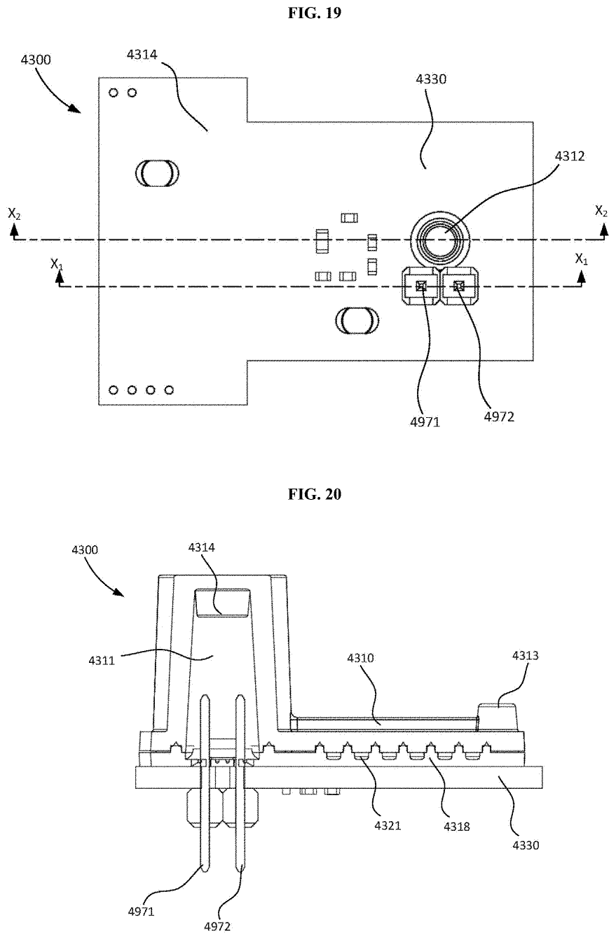

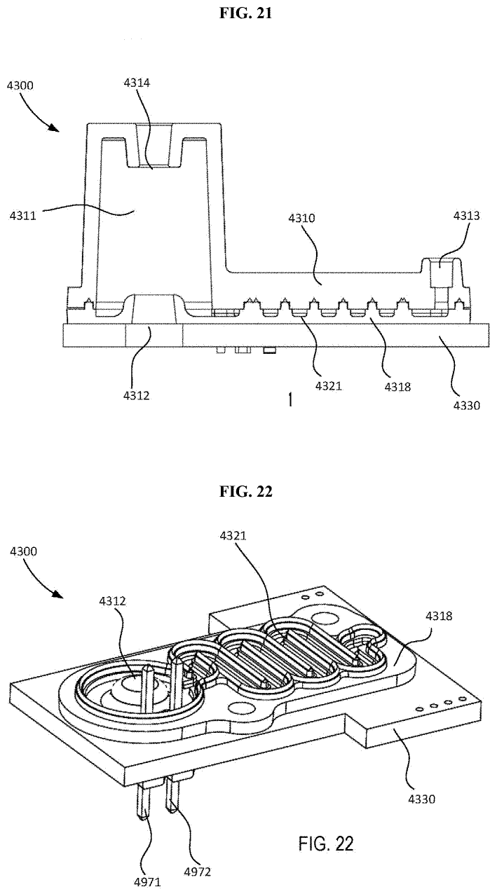

FIGS. 17-22 show various views of a lysing module 4300 (also referred to as an inactivation module), according to an embodiment. The lysing module 4300 includes a chamber body 4310, a bottom lid 4318, a heater 4330, and an electrode assembly. The chamber body 4310 and the bottom lid 4318 can be referred to as a flow member. Although the flow member is shown as being constructed from two pieces (the body 4310 and the bottom lid 4318) that are coupled together, in other embodiments, the flow member can be monolithically constructed. The chamber body 4310 and the bottom lid 4318 define an input port 4312, a first (or holding) volume 4311, a vent 4314, a second (or inactivation) volume 4321, and an output port 4313. The input port 4312 can receive the eluent from the elution chamber and/or directly from a filter assembly (e.g., the filter assembly 1230). In other embodiments, as described herein, the input port 4312 can be fluidically coupled to a sample input module without the biological input being conveyed through a filter. In use, the eluent can flow into the lysing module 4300 and be collected in the holding volume 4311. The sample can be lysed within the holding volume 4311. For example, in some embodiments, the eluent containing the target organisms can be heated by the heater 4330 to maintain the eluent at or above a target lysing temperature. Similarly stated, in some embodiments, the heater 4330 can be coupled to the chamber body 4310 and/or the bottom lid 4318 such that the heater 4330 can convey thermal energy into the lysing module 4300 to produce a lysing temperature zone within the holding volume 4311. The lysing temperature zone can maintain the eluent at any of the temperatures and for any of the time periods described herein.

The vent opening 4314 is in fluid communication with the first volume 4311, and thus allows air to flow into or out of the lysing module 4300 (including the first volume 4311 and the second volume 4321) as sample is conveyed into and/or out of the lysing module 4300. The vent 4314 can also relieve pressure within either of the first volume 4311 or the second volume 4321 when the eluent is heated. Although shown as being a permanent vent (i.e., a vent having a fixed opening), in some embodiments, the lysing module 4300 (or any of the lysing modules described herein) can have an active vent. For example, in some embodiments, the lysing module 4300 (or any of the lysing modules described herein) can include a valve that controls the venting of pressure and/or air from within the lysing module 4300.

The first volume 4311 is in fluid communication with the second volume 4322. In this manner, the eluent can flow from the first (or holding) volume 4311 through the second (or inactivation) volume 4321 of the lysing module 4300. More specifically, when a pressure gradient is applied across the input port 4312 and the output port 4313, the eluent can flow from the holding volume 4311 (first volume) through the second volume 4322. The pressure gradient can be applied by any suitable mechanism, such as for example, a pump (e.g., the fluidic drive module 1400). As shown, the second volume 4321 is a serpentine channel that provides a high surface area to volume ratio. This arrangement allows for rapid inactivation of the lysis enzymes in the eluent. The eluent, after being flowed through the inactivation segment, may be flowed into the output port 4313 to be collected and/or conveyed to an amplification module (not shown).

As described above, the flow member is in contact with a heating element 4330, which can be, for example, a printed circuit board (PCB) heater. The heating element 4330 may function to heat the eluent as it flows through the second volume 4311 at a high temperature sufficient to inactivate the one or more lysis enzymes contained within the eluent. For example, the heating element may heat the eluent to about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C. By heating the liquid eluent to a high temperature, the lysis enzymes as well as any other enzymes present can be deactivated. In some embodiments, the sample can be heated to about 95 C for about 4 minutes.

In some embodiments the heater on the PCB 4330 is specifically designed to heat the serpentine portion of the lysing module 4300 (i.e., the second volume 4321) while not heating the holding volume 4311. Because the lid 4318 of the lysing module 4300 is thick, the heater surface may be heated well above the desired temperature of the fluid. Since the electrodes 1971, 1972 (described in more detail below) are thermally conductive and come into direct contact with the fluid, the fluid surrounding the electrodes 1971, 1972 will experience the same temperature as the heater surface, which may cause evaporation. To minimize the heating of the holding volume 4311, a slot (not shown) may be cut in the PCB 4330 to isolate the heater from the portion of the PCB adjacent and/or in contact with the holding volume 4311. For example, in some embodiments, the heater 4330 can include a series of slots and/or openings as described in U.S. patent application Ser. No. 15/494,145, entitled “Printed Circuit Board Heater for an Amplification Module,” which is incorporated herein by reference in its entirety. Moreover, in some embodiments, the heating element of the heater 4330 is located on an internal layer so the top copper pour (not shown) can be used as a heat spreader to minimize temperature variation along the serpentine path. The six wires soldered to the PCB 4330 may remove heat from the surrounding area, creating temperature gradients across the heater surface. To minimize this effect, wires may be soldered on both sides of the heater surface so the temperature roll off is symmetrical.

In some embodiments, the lysing module 4300 can determine whether there is liquid in the first volume 4311 and/or the second volume 4321. Specifically, the lysing module 4300 includes electrical probes to determine electrical resistance of the fluid within the first volume. In some embodiments, the molecular diagnostic device (e.g., the device 1000) can include an electronic controller configured to determine when the user has actuated the elution module (e.g., by pressing an elution actuator, similar to the button 1070 described above) by detecting the presence of liquid in the first volume 4311. In this manner, the introduction of liquid into the first volume 4311 can trigger the start of the device.

Specifically, the control system and/or the lysing module 4300 includes two electrodes 4971, 4972 inside the first volume 4311. The electrodes 4971, 4972 are connected to circuitry (e.g., a controller, not shown) that detects a resistance change between the two electrodes 4971, 4972. Fluid may be reliably detected between the electrodes 4971, 4972 due to the high gain of the circuit, which may easily differentiate between an open circuit condition (no fluid) and a non-negligible resistance across the electrodes 4971, 4972 (fluid detected). Use of a sample matrix with high salt concentration increases the conductivity of the fluid, which may make the fluid easily detectable even with variation across samples.

The electrodes 4971, 4972 and the circuitry (not shown) are designed to detect fluid without impacting the biological processes that take place in the device. For example, the electrodes 4971, 4972 are specifically chosen so as not inhibit PCR reactions. In some embodiments, the electrodes 4971, 4972 are gold plated.

Both DNA and cells have a net charge so they may migrate in the presence of an electric field. Because the resistance change between the electrodes 4971, 4972 is determined by measuring a change in electric potential, precautions may be taken to minimize the impact of this electromotive force. For example, once fluid is detected voltage may be removed from the electrodes 4971, 4972 and they may be electrically shorted together. This ensures there is no potential difference between the electrodes 4971, 4972 and the charged particles (DNA, cells, salts, etc.) will not bind to the electrodes, which would prevent them from entering the amplification module (not shown).

As described, the solution within the second volume 4321 is rapidly heated to temperatures of up to about 100 degrees Celsius. The lysing module 4300 and/or the formulation of the input solution (e.g., the eluent), however, can collectively reduce the likelihood that the liquid portion of the input solution will boil during the lysing/inactivation operations. Such boiling can produce undesirable bubbles and/or air pockets and can reduce the repeatability of the lysing and/or inactivation operations. Moreover, to facilitate use of the device at a variety of different altitudes, the lysing module 4300 and/or the formulation of the input solution can collectively reduce the likelihood that the liquid portion of the input solution will boil at a temperature of 99 degrees Celsius or higher, 98 degrees Celsius or higher, 96 degrees Celsius or higher, 94 degrees Celsius or higher, 92 degrees Celsius or higher, 90 degrees Celsius or higher, or 88 degrees Celsius or higher. For example, in some embodiments, the input solution can include salts and/or sugars to raise the boiling temperature of the input solution. In other embodiments, the lysing module 4300 can include one or more vent openings into either the first volume 4311 or the second volume 4321 or both (to limit pressure build-up during heating).

The reverse transcription module consists of an incubation chamber in which a reverse transcription reaction can take place and a means to heat the sample to a temperature sufficient to deactivate a reverse transcriptase enzyme. The reverse transcriptase may be present as a lyophilized pellet in the incubation chamber of the reverse transcription module. The lyophilized pellet is rehydrated by the sample when the sample enters 1900, thus allowing a reverse transcription reaction to occur. The lyophilized pellet may contain suitable salts to buffer the sample to ensure suitable conditions for the reverse transcriptase enzyme. In some cases the reverse transcription enzyme may be chosen to have activity in the sample without requiring additional buffers. The lyophilized pellet may also contain compounds of additives to stabilize the enzyme in the lyophilized state and preserve enzymatic activity once rehydrated. The lyophilized pellet may contain primers for the reverse transcriptase enzyme. The primers may be specific primers to amplify RNA molecules of specific sequences, random primers such as random hexamers, or primers targeted to common sequences, such as poly T primers to amplify RNA molecules with poly-A tails.

The reverse transcription reaction may occur in the incubation chamber of the reverse transcription module 1900. The incubation chamber may be

After the lysing and inactivation operations, the output from the lysing module 4300 can be conveyed into an (e.g., the amplification module 1600 or any other amplification modules described herein). Similarly stated, the output from the lysing module 4300, which contains the extracted nucleic acid molecules, can be conveyed to an amplification module. The amplification module can then perform a thermal reaction (e.g., an amplification reaction) on the prepared solution containing target nucleic acid mixed with required reagents. In some embodiments, the amplification module is configured to conduct rapid amplification of an input target. In some embodiments, the amplification module is configured to generate an output copy number that reaches or exceeds the threshold of the sensitivity of an associated detection module (e.g., the detection module 1800).

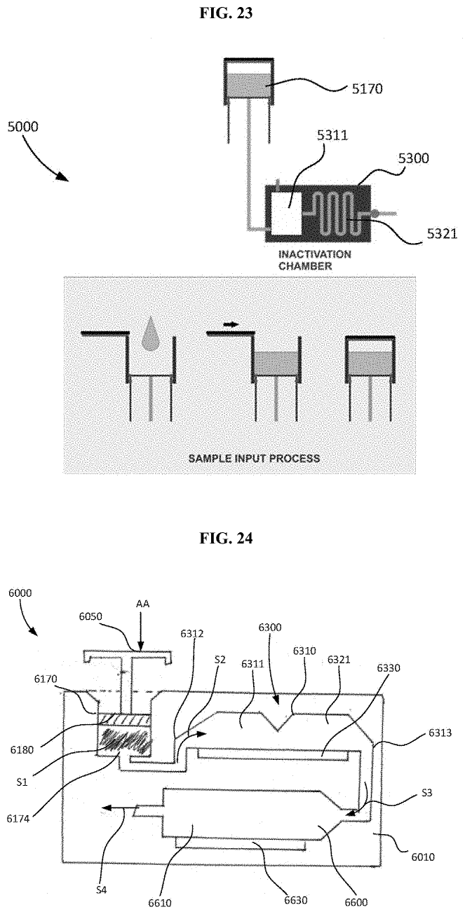

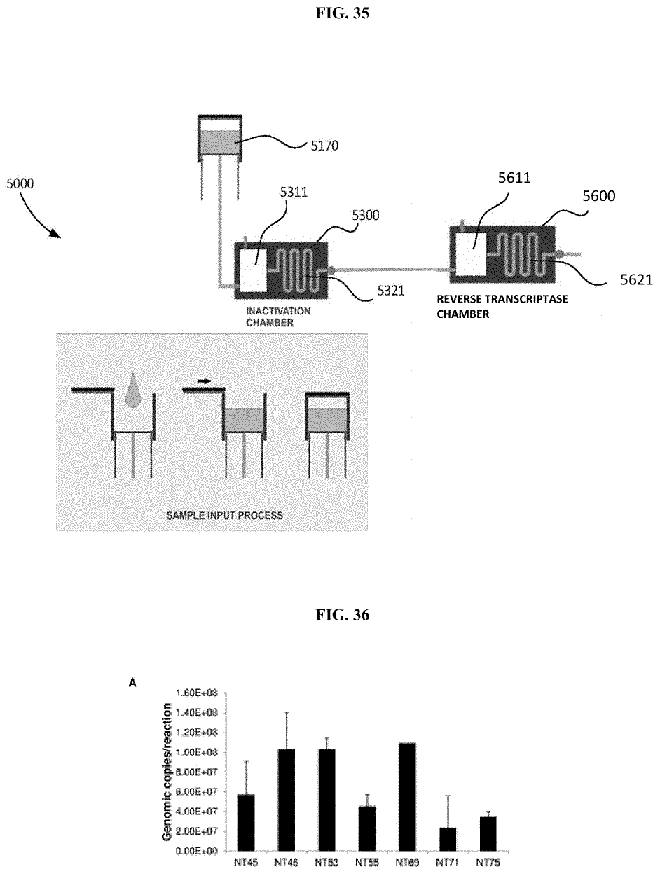

Although the device shown in FIG. 10 is described as including a filter assembly, in some embodiments, a sample preparation device need not include a filter or filter assembly. For example, in some embodiments the sample input may be directly linked to an inactivation chamber, as shown schematically in FIG. 23. Advantages of a device without a filter assembly include lower pressures in the device, no risk of breaking a filter, fewer parts, fewer reagents required, higher recovery of target organisms from the clinical sample matrix and higher recovery of DNA from target organisms. FIG. 23 and FIG. 35 shows a portion of a molecular test device 5000 that includes a sample input module 5170 and an inactivation (or lysing) module 5300. The portion of a molecular test device in FIG. 35 further comprises a reverse transcription module 5600. The device 5000 can be similar to the device 1000 described above, and can include an amplification module, a detection module or the like. In this case, the device 5000 differs from the device 1000 in that the sample is flowed from the input module 5170 into the holding tank of the inactivation module 5300. The sample may be lysed either in the holding tank 5311 or in the inactivation segment 5321. In this case the sample may be lysed by heating without need for a specialized lysis buffer or lysis enzymes. Any proteases or nucleases released from the cells of the sample will be inactivated by heating. For example, a sample may be flowed into the holding tank and held until the inactivation segment 5321 reaches a set temperature (for example greater than 90 C) and then flowed through the inactivation segment. In the inactivation segment the sample is rapidly heated to 95 C causing the cells in the sample to lyse and proteins from within the cells to be inactivated. The sample may be reverse transcribed in the reverse transcription chamber 5611 and the reverse transcriptase enzyme may be inactivated in the inactivation segment 5621.

As another example of an embodiment in which the sample is not conveyed through a filter, FIG. 24 is a schematic illustration of a molecular diagnostic test device 6000 (also referred to as a “test device” or “device”), according to an embodiment. The test device 6000 includes a housing 6010, a sample input module 6170, a lysing module 6300, and an amplification module 6600. The housing 6010 can be any structure within which the sample input module 6170, the lysing module 6300, and the amplification module 6600 are contained. In some embodiments, the test device 6000 can have a size, shape and/or weight such that the device can be carried, held, used and/or manipulated in a user's hands (i.e., it can be a “handheld” device). In other embodiments, the test device 6000 can be a self-contained, single-use device of the types shown and described herein (e.g., the device 1000) or in International Patent Publication No. WO2016/109691, entitled “Devices and Methods for Molecular Diagnostic Testing,” which is incorporated herein by reference in its entirety.

The sample input module 6170 is disposed within the housing 6010, and is configured receive a biological sample S1 containing a biological entity. The biological sample S1 can be any of the sample types described herein, and the biological entity can be any of the entities described herein. The sample input module 6170 defines a sample volume 6174, and includes a piston 6180 that is movably disposed within the sample volume 6174. In use the biological sample S1 can be conveyed into the sample volume 6174 by any suitable mechanism, such as, for example, via a pipette, a dropper, or the like. In some embodiments, the biological sample S1 can be conveyed via an opening into the sample volume 6174 that can be blocked to prevent backflow of the sample back out of the sample input volume 6174. For example, in some embodiments, the sample input module 6170 can include any suitable flow control devices, such as check valves, duck-bill valves, or the like, to control the flow of the biological sample S1 within the device 6000.

The sample input module 6170 (and any of the sample input modules described herein) can be actuated by any suitable mechanism to convey the biological sample S1 towards the lysing module 6300 to enable the nucleic acid extraction methods described herein. For example, in the embodiment shown, the sample input module 6170 is actuated by the sample actuator (or button) 6050. The sample actuator 6050 is movably coupled to the housing 6010, and is aligned with and can move the piston 6180 when the sample input module 6170 is actuated. The sample actuator 6050 is a non-electronic actuator that is manually depressed by a user to actuate the sample input module 6170. In other embodiments, however, the sample actuator 6050 can be an electronic actuator. In some embodiments, the sample actuator 6050 can include a lock tab (not shown) that is fixedly received within the notch or opening of the housing 6010 to fix the sample actuator 6050 in its second or “actuated” position, as described above. In this manner, the device 6000 cannot be reused after the initial actuation. When the piston 6180 is moved downward within the sample volume 6174, as shown by the arrow AA, the sample within the sample volume 6174 is conveyed towards the lysing module 6300. The flow of the biological sample S1 towards the lysing module 6300 is shown by the arrow S2 in FIG. 24.

The lysing module 6300 (also referred to as the inactivation module), which can be a portion of a sample preparation module, is configured to process the biological sample S1 to facilitate detection of an organism therein that is associated with a disease. Specifically, the lysing module 6300 is configured to concentrate and lyse cells in the biological sample S1, thereby allowing subsequent extraction of a nucleic acid to facilitate amplification (e.g., via the amplification module 6600) and/or detection (e.g., via a detection module, not shown). As shown, the processed/lysed sample (e.g., the sample S3) is pushed and/or otherwise transferred from the lysing module 6300 to other modules within the device 6000 (e.g., the amplification module 6600). By eliminating the need for external sample preparation and a cumbersome instrument, the device 6000 is suitable for use within a point-of-care setting (e.g., doctor's office, pharmacy or the like) or at the user's home, and can receive any suitable biological sample S1. The biological sample S1 (and any of the input samples described herein) can be, for example, blood, urine, male urethral specimens, vaginal specimens, cervical swab specimens, and/or nasal swab specimens gathered using a commercially available sample collection kit.

The lysing module includes a flow member 6310 and a heater 6330. The flow member 6310 includes an input port 6312 and an output port 6313, and defines a first volume 6311 and a second volume 6321. As shown, the first volume 6311 can receive an input solution (identified as S2) containing at least the biological sample S1 and a lysis buffer. The lysis buffer can be any of the lysis buffers described herein. Moreover, the lysis buffer can be mixed with the biological sample S1 to form the input solution S2 in any suitable manner or at any suitable location within the device 6000. For example, in some embodiments, the lysis buffer can be stored within the sample input module 6170, and can be mixed with the biological sample S1 when the biological sample S1 is conveyed into the volume 6174. In other embodiments, the lysis buffer can be stored in a reagent module (not shown) and can be mixed with the biological sample S1 when the sample input module 6170 is actuated (e.g., via the actuator 6050). In yet other embodiments, the lysis buffer can be stored in the lysing module 6300 (e.g., the first volume 6311).

The heater 6330 is coupled to the flow member 6310 and is configured to produce thermal energy that is conveyed into the first volume 6311, the second volume 6321, or both the first volume 6311 and the second volume 6321 to lyse organisms within the biological sample S1 and/or the input solution S2. In this manner, the lysing module 6300 can release one or more nucleic acid molecules from within the cells and/or organisms within the biological sample S1 and/or the input solution S2. Specifically, the heater 6330 and the flow member 6310 are collectively configured to maintain the input solution S2 at a desired lysing temperature for a predetermined amount of time to facilitate and/or promote lysing of the organisms therein. For example, in some embodiments, the first volume 6311 and/or the second volume 6321 can be maintained at a temperature between about 55 degrees Celsius and about 600 degrees Celsius for a time period of about 25 seconds or more. In other embodiments, the first volume 6311 and/or the second volume 6321 can be maintained at a temperature between about 92 degrees Celsius and about 98 degrees Celsius.

In addition to lysing organisms within the input solution S2 to release nucleic acid molecules, the heater 6330 and the flow member 6310 are configured to heat the first volume 6311, the second volume 6321, or both the first volume 6311 and the second volume 6321 to inactivate enzymes present within the biological sample S1 and/or the input solution S2. Specifically, by heating the input solution S2, the lysing module 6300 can denature certain proteins and/or inactivate certain enzymes present within organisms that are within the input solution S2. Such proteins and/or enzymes can, in certain instances, limit the efficiency or effectiveness of the desired amplification operation. Thus, rapid and efficient inactivation can improve the repeatability and accuracy of the amplification and/or the detection of the molecular diagnostic device 6000. In some embodiments, for example, the heater 6330 and the flow member 6310 can collectively produce an inactivation temperature zone within which the input solution S2 can be heated to within the desired temperature range and/or for the desired time period to produce the desired inactivation. For example, in some embodiments, the input solution S2 within the lysing module 6300 can be maintained at a temperature between about 55 degrees Celsius and about 600 degrees Celsius for a time period of about 25 seconds or more. In other embodiments, the input solution S2 within the lysing module 6300 can be maintained at a temperature between about 92 degrees Celsius and about 98 degrees Celsius.

Although described as occurring in two separate heating operations, the lysing and the inactivation can be performed by a single heating operation. For example, in some embodiments, the input solution S2 can be heated to the desired temperature range to both lyse the organisms and inactivate the enzymes as the input solution S2 flows through the first volume 6311 and/or the second volume 6321. Said another way, in some embodiments, the lysing module 6300 can perform “flow through” inactivation and lysing operations. For example, in some embodiments, either of the first volume 6311 or the second volume 6321 (or both) can define a tortuous flow path through which the input solution S2 flows during the lysing/inactivation operation. In this manner, the surface area-to-volume ratio of the first volume 6311 and/or the second volume 6321 can be high enough such that the heat transfer into the input solution S2 occurs rapidly as it flows through the lysing module. In some embodiments, for example, the first volume 6311 and/or the second volume 6321 can define a serpentine flow path. In some embodiments, a ratio of the surface area of the second volume 6321 to the volume of the second volume 6321 is 20 cm⁻¹.

In some embodiments, the flow member 6310 (and any of the flow members described herein) can have a volume about 650 microliters or greater, and the flow can be such that at least 60 microliters of the input solution S2 is prepared for amplification (i.e., has nucleic acids extracted therefrom). In other embodiments, at least 20 microliters of the input solution S2 is prepared for amplification by the methods and devices described herein. In other embodiments, at least 30 microliters of the input solution S2 is prepared for amplification by the methods and devices described herein. In yet other embodiments, at least 50 microliters of the input solution S2 is prepared for amplification by the methods and devices described herein.

As described above, in some embodiments, the input solution S2 is rapidly heated to temperatures of up to about 100 degrees Celsius. The lysing module 6300 and/or the formulation of the input solution S2, however, can collectively reduce the likelihood that the liquid portion of the input solution S2 will boil during the lysing/inactivation operations. Such boiling can produce undesirable bubbles and/or air pockets and can reduce the repeatability of the lysing and/or inactivation operations. Moreover, to facilitate use of the device at a variety of different altitudes, the lysing module 6300 and/or the formulation of the input solution S2 can collectively reduce the likelihood that the liquid portion of the input solution S2 will boil at a temperature of 99 degrees Celsius or higher, 98 degrees Celsius or higher, 96 degrees Celsius or higher, 94 degrees Celsius or higher, 92 degrees Celsius or higher, 90 degrees Celsius or higher, or 88 degrees Celsius or higher. For example, in some embodiments, the input solution S2 can include salts and/or sugars to raise the boiling temperature of the input solution S2. In other embodiments, the lysing module 6300 can include one or more vent openings into either the first volume 6311 or the second volume 6321 or both (to limit pressure build-up during heating). In such embodiments, the vent opening can be such that a limited amount of pressure is allowed within the first volume 6311 or the second volume 6321 to raise the boiling temperature of the input solution S2.

After the lysing and inactivation operations, the output from the lysing module 6300 can be conveyed into the amplification module 6600. Similarly stated, the output from the lysing module 6300, which is identified as the prepared solution S3, and which contains the extracted nucleic acid molecules, can be conveyed to the amplification module 6600. The amplification module 6600 can then perform a thermal reaction (e.g., an amplification reaction) on the prepared solution S3 containing target nucleic acid mixed with required reagents. In some embodiments, the amplification module 6600 is configured to conduct rapid amplification of an input target. In some embodiments, the amplification module 6600 is configured to generate an output copy number that reaches or exceeds the threshold of the sensitivity of an associated detection module.

The amplification module 6600 includes a flow member 6610 and a heater 6630. The flow member 6610 can be any suitable flow member that defines a volume or a series of volumes within which the prepared solution S3 can flow and/or be maintained to amplify the target nucleic acid molecules within the solution S3. The heater 6630 can be any suitable heater or group of heaters coupled to the flow member 6610 that can heat the prepared solution S3 within the flow member 6610 to perform any of the amplification operations as described herein. For example, in some embodiments, the amplification module 6600 (or any of the amplification modules described herein) can be similar to the amplification modules shown and described in U.S. Patent Application No. 65/494,145, entitled “Printed Circuit Board Heater for an Amplification Module,” which is incorporated herein by reference in its entirety.

In some embodiments, the flow member 6610 defines a single volume within which the prepared solution S3 is maintained and heated to amplify the nucleic acid molecules within the prepared solution S3. In other embodiments, the flow member 6610 can define a “switchback” or serpentine flow path through which the prepared solution S3 flows. Similarly stated, the flow member 6610 defines a flow path that is curved such that the flow path 6618 intersects the heater 6630 at multiple locations. In this manner, the amplification module 6600 can perform a “flow through” PCR where the prepared solution S3 flows through multiple different temperature regions.

The flow member 6610 (and any of the flow members described herein) can be constructed from any suitable material and can have any suitable dimensions to facilitate the desired amplification performance for the desired volume of sample. For example, in some embodiments, the amplification module 6600 (and any of the amplification modules described herein) can perform 6000× or greater amplification in a time of less than 65 minutes. For example, in some embodiments, the flow member 6610 (and any of the flow members described herein) is constructed from at least one of a cyclic olefin copolymer or a graphite-based material. Such materials facilitate the desired heat transfer properties into the flow path 6620. Moreover, in some embodiments, the flow member 6610 (and any of the flow members described herein) can have a thickness of less than about 0.5 mm. In some embodiments, the flow member 6610 (and any of the flow members described herein) can have a volume about 150 microliters or greater, and the flow can be such that at least 10 microliters of sample is amplified. In other embodiments, at least 20 microliters of sample are amplified by the methods and devices described herein. In other embodiments, at least 30 microliters of sample are amplified by the methods and devices described herein. In yet other embodiments, at least 50 microliters of sample are amplified by the methods and devices described herein.

The heater 6630 can be any suitable heater or collection of heaters that can perform the functions described herein to amplify the prepared solution S3. In some embodiments, the heater 6630 can establish multiple temperature zones through which the prepared solution S3 flows and/or can define a desired number of amplification cycles to ensure the desired test sensitivity (e.g., at least 30 cycles, at least 34 cycles, at least 36 cycles, at least 38 cycles, or at least 40 cycles). The heater 6630 (and any of the heaters described herein) can be of any suitable design. For example, in some embodiments, the heater 6630 can be a resistance heater, a thermoelectric device (e.g. a Peltier device), or the like. In some embodiments, the heater 6630 can be one or more linear “strip heaters” arranged such that the flow path crosses the heaters at multiple different points. In other embodiments, the heater 6630 can be one or more curved heaters having a geometry that corresponds to that of the flow member 6610 to produce multiple different temperature zones in the flow path.

Although the amplification module 6600 is generally described as performing a thermal cycling operation on the prepared solution S3, in other embodiment, the amplification module 6600 can perform any suitable thermal reaction to amplify nucleic acids within the solution S3. In some embodiments, the amplification module 6600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process.

In some embodiments, a molecular diagnostic test device includes a reverse transcription (RT-PCR) module, which may be positioned between the lysis module and the amplification module.

Reverse transcription (RT) is the process of converting RNA into cDNA. One of the main reasons to do this conversion is that the subsequent cDNA can be amplified in PCR. The best way to convert RNA into cDNA is by using an enzyme called Reverse Transcriptase. This enzyme, however is most efficient by itself before a PCR reaction, due to its temperature and buffering needs. However, there are instances where the RT-PCR and PCR reactions are conducted in the same tube. This requires a mix of both Reverse Transcriptase and DNA Polymerase.

In some embodiments, a sample containing RNA, or suspected of containing RNA, is delivered from the sample prep subsystem into a chamber that contains a dried or lyophilized pellet. This pellet contains dried or lyophilized Reverse Transcriptase enzyme, dried or lyophilized reverse transcriptase reagents, and possibly the salts needed to create the correct buffering environment for the RT-PCR. The pellet dissolves in the solution containing RNA and is held at a constant temperature (somewhere between 20° C. and 50° C.) for some period of time (from 0.1 seconds to 24 hours). During this incubation cDNA is produced from the RNA in the eluted sample.

The subsequent cDNA solution can then be heated at an elevated temperature (50° C. to 100° C.) for some time period (from 0.1 seconds to 24 hours) to inactivate the RT-PCR enzyme. After mixing the solution is now ready for PCR. The device will flow the ready cDNA solution into a mixing chamber containing reagents for PCR, followed by subsequent PCR and detection as described elsewhere in this application.

In another embodiment, RNA is delivered from the sample prep subsystem straight into a mixing chamber that contains dried or lyophilized reagents for one-step RT-PCR. This one-step RT-PCR reaction may be done either because a special enzyme is used that can do both the RT-PCR and conventional PCR tasks, or it is done by a mixture of both RT and DNA polymerase. After mixing (and possibly incubating at 30-60° C. for 0.1 second to 1 hour) the solution is now ready for PCR. The reaction is processed through subsequent PCR and detection.

Note that other amplification methods other than PCR, such as isothermal amplification could also be used with the cDNA solution produced by the RT-PCR reaction.

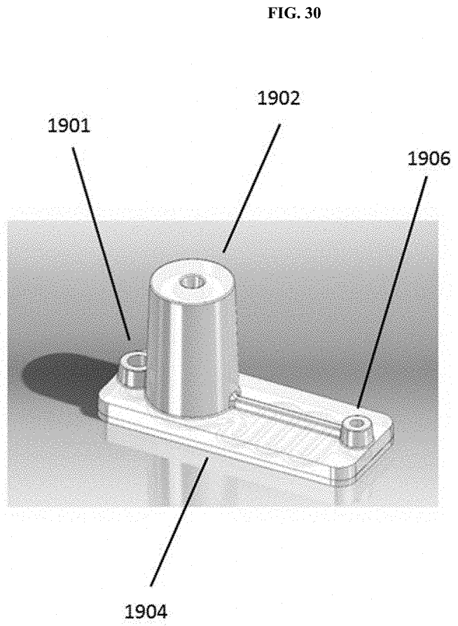

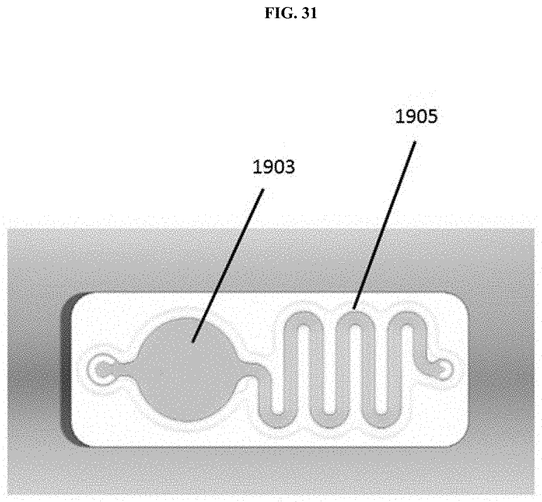

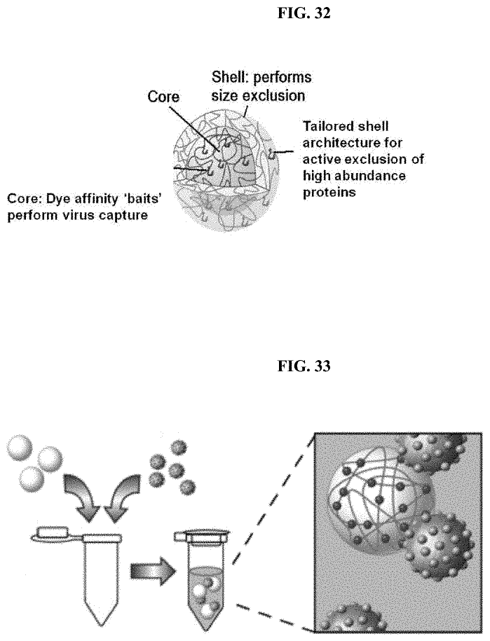

One possible embodiment of the RT module is shown in FIG. 30 and FIG. 31. RNA elution volume may enter port 1901 and flow into chamber 1902 designed to hold approximately 300 ul of fluid. Chamber 1902 holds a lyophilized pellet consisting of suitable RT-PCR reagents. Heater 1904 heats the bottom of the assembly, both the holding chamber (1903) and the serpentine channel (1905). The chamber is elevated to a temperature T_{RT}, between 20 C and 50 C, which is optimal for the RT reaction. The entering fluid hydrates the lyophilized pellet. The liquid in chamber (1903) is incubated for time t₁ (0.1 to 24 hours) and then the chamber and serpentine flow channel is elevated to T_{inact}, (85-95 C) a temperature suitable for inactivation of inhibiting reagents. At this point a flow is caused by a vacuum or positive pressure to move the fluid from the holding chamber (1902, 1903) through a serpentine channel (1905) to a port 1906 where fluid exits to the next step. The serpentine channel is designed to have a cross-section with an aspect ratio (channel height to width) to maximize the area in contact with heater allowing efficient heat coupling to the fluid. The flow rates in the channel are set to achieve a minimum dwell time in the channel to achieve reagent inactivation.

In some embodiments, the RT module may be identical to an inactivation module described herein. In some embodiments, the RT module may be identical to an inactivation module described herein, expect for the presence of lyophilized RT enzyme and other components required for the RT reaction. In some embodiments the RT module may resemble any one or more of the inactivation modules shown in FIGS. 13-24.

In some embodiments the RT module and the inactivation module may be the same module. The inactivation and RT module may comprise two output ports, a first output port which leads into a chamber which contains a lyophilized RT enzyme and then connects back to the input port of the module, and a second output port which leads to the mixing chamber. The first output port may connect back to the input port via a one way valve.

The devices described herein may include and/or be coupled to an amplification module or PCR module of the types shown and described herein, in which a polymerase chain reaction may be performed. The amplification module may be proceeded by a mixing chamber in which the nucleic acid is mixed with components for performing a polymerase chain reaction. Examples of components which may be required for a polymerase chain reaction include nucleotide triphosphates, polymerase enzymes, nucleic acid primers, calcium ions and buffer. In some examples, all components of the reaction mixture may be present in the sample buffer. In other examples the sample buffer may comprise all components except for a polymerase enzyme which may be provided in the mixing chamber. The choice of polymerase enzyme may depend on the purification and lysis protocol used. In some examples, the devices may also comprise a detection module which is capable of detecting nucleic acids amplified in the amplification module.

The devices described herein may be contained with a housing. In some cases, the device is self-contained. In some cases, the device is a handheld device. In some cases, the device is configured for one-time use (e.g., disposable). In some instances, the devices may generate a nucleic acid sample that may be collected prior to performing one or more downstream applications. For example, the sample can be held in a chamber or reservoir within the housing of the device or can be relayed to a chamber or reservoir that sits outside of the housing of the device. In other examples, the device is coupled to one or more additional devices that can perform the one or more downstream applications, for example, a device that can perform a polymerase chain reaction (PCR).

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Comparison of a Traditional DNA Extraction Method Versus an Embodiment of the Methods Described Herein

In this example, DNA was extracted from clinical samples using either a standard DNA extraction protocol or a DNA extraction protocol using the methods described herein. Clinical samples that were positive for Neisseria gonorrhoeae and/or Chlamydia trachomatis (Samples 101, 105, 108, 117 and 122) were obtained and screened for the presence of these bacteria (See Table 1). These samples were processed utilizing two different methods for DNA extraction. For the first method, 500 μL of each of these samples were taken for DNA extraction utilizing the Qiagen QIAmp® DNA Mini Kit according to the manufacturer's recommendations for isolation of bacterial DNA from bodily fluids (“standard method”). For the second method, 500 of each of the samples were taken for DNA extraction utilizing an embodiment of the methods provided herein. Briefly, 500 μL of the sample was preloaded into a clean syringe and 1 mL of air was aspirated into the same syringe. The syringe containing both the sample and air was connected to the filter housing and the entire volume was pushed through (i.e., liquid followed by air). A new syringe was preloaded with 600 μL of wash solution, then the wash solution was pushed through the filter housing. The orientation of the filter was flipped and a female luer lug was attached to the end. Using a new syringe, 350 μL of TT buffer (Tris Acid, Tris Base, Tween 80, Antifoam SE-15, ProClin™300 and molecular grade water) was pushed through the filter in order to elute the sample off the filter into a 1.5 mL tube. The 1.5 mL tube was preloaded with a lyophilized proteinase K pellet. The tube was incubated in a heat block at 56° C. for 1 minute to allow for optimal proteinase K activity. The proteinase K was heat inactivated by placing the tube in a heat block at 95° C. for 10 minutes.

TABLE 1

| | | Purification |
|---|---|---|
| Condition | Sample | Method |
| 1 | 105 | Qiagen |
| 2 | 117 | Qiagen |
| 3 | 101 | Qiagen |
| 4 | 108 | Qiagen |
| 5 | 122 | Qiagen |
| 6 | 105 | Click SP |
| 7 | 117 | Click SP |
| 8 | 101 | Click SP |
| 9 | 108 | Click SP |
| 10 | 122 | Click SP |
| 11 | Positive Control | N/A |
| 12 | No template | N/A |
| | control (water) | |

Each sample was mixed with PCR reagents. Primer/probe sets designed to amplify sequences from several different organisms were added to each sample. 1 μL of N. subflava DNA (1,000 copies/rxn) were added to the sample/PCR mix designated for the NS assay. The mixtures were divided into two wells of 20 μL each on a LightCycler® plate. The plate was loaded onto the LightCycler® Real-Time PCR System (Roche) and run under the following PCR conditions:

Stage 1: 95 C for 20 seconds
Stage 2: 40 cycles of: 95 C for 1 second, 60 C for 6 seconds

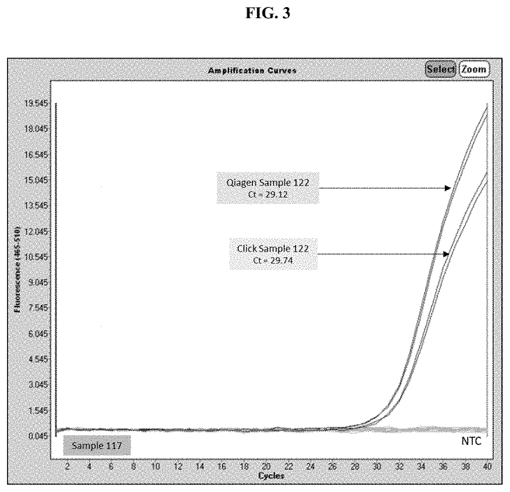

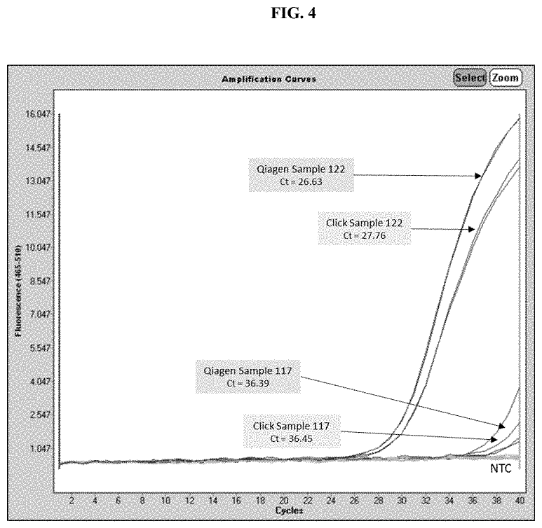

FIGS. 3 and 4 depict a comparison of data generated from real-time PCR reactions performed on DNA extracted from a clinical sample positive for both N. gonorrhoeae and C. trachomatis (Sample 122) and a clinical sample positive for N. gonorrhoeae (Sample 117) utilizing the methods provided herein versus standard DNA extraction methods. Primer Set #1 detected the presence of N. gonorrhoeae in Sample 122 prepared using either method as shown in FIG. 3. Primer Set #2 detected the presence of N. gonorrhoeae in both Sample 122 and Sample 117, prepared using either method as shown in FIG. 4. Both the standard (“Qiagen sample”) and the new method (“Click sample”) yielded a Ct value of ˜36 with an endpoint signal of less than 5, indicating that the sample had a low titer of N. gonorrhoeae. (FIG. 4)

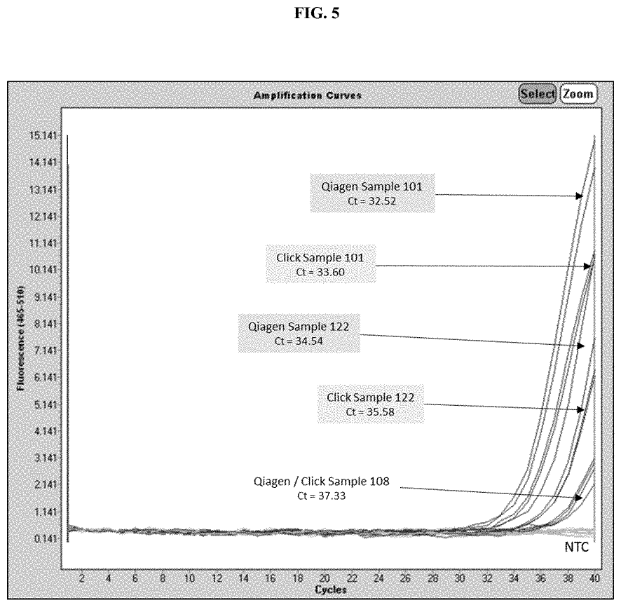

FIGS. 5 and 6 depict a comparison of data generated from real-time PCR reactions performed on DNA extracted from a clinical sample positive for both N. gonorrhoeae and C. trachomatis (Sample 122), and clinical samples positive for C. trachomatis (Samples 101 and 108) utilizing the methods provided herein versus standard DNA extraction methods. Both standard (“Qiagen”) and new methods (“Click”) of DNA extraction did not detect the presence of C. trachomatis in Sample 105 using either Primer Set #3 or Primer Set #4. Primer Set #3 was able to detect the presence of C. trachomatis in Samples 108, 122 and 101 using either sample preparation method (FIG. 5). Primer Set #4 was able to detect the presence of C. trachomatis in Sample 101 for both sample preparation methods, and only Sample 122 for the standard method, and only Sample 108 for the new method (FIG. 6).

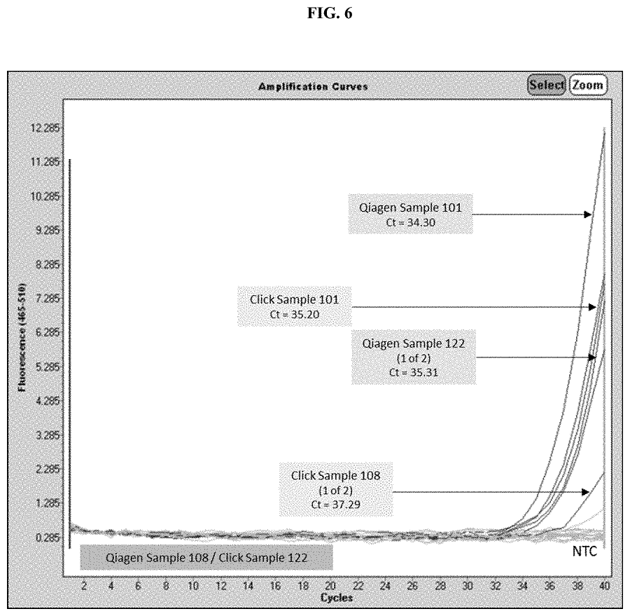

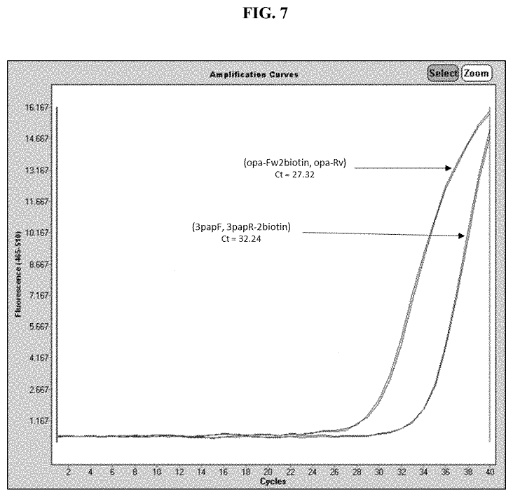

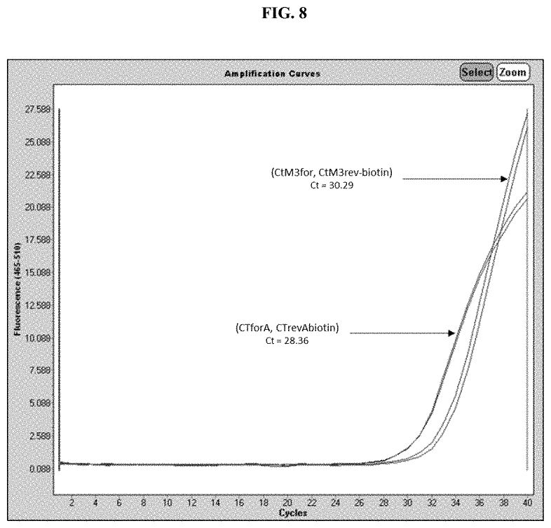

FIGS. 7 and 8 depict a comparison of data generated from real-time PCR reactions performed on N. gonorrhoeae positive control DNA or C. trachomatis positive control DNA, respectively, utilizing different sets of primers.

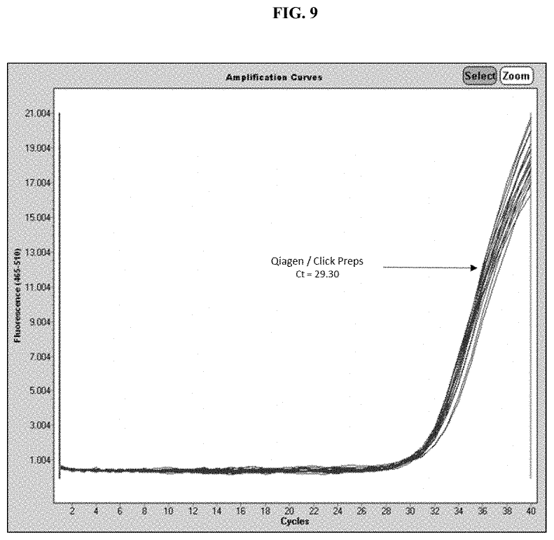

FIG. 9 depicts data generated from a real-time PCR reaction performed on N. gonorrhoeae DNA spiked into a sample and PCR mixture to test for sample inhibition.

### Example 2. PCR Amplification from Samples Purified without a Filter Step

In this example DNA was purified from a range of samples using the no filter method described herein. Briefly samples are flowed into the holding chamber of the inactivation module. the heat-treated fluid is flowed through the serpentine path and into a mixing chamber containing PCR reagents. PCR is performed and PCR products are detected. In this example, purified DNA is subjected to PCR using the probe sets of example 1.

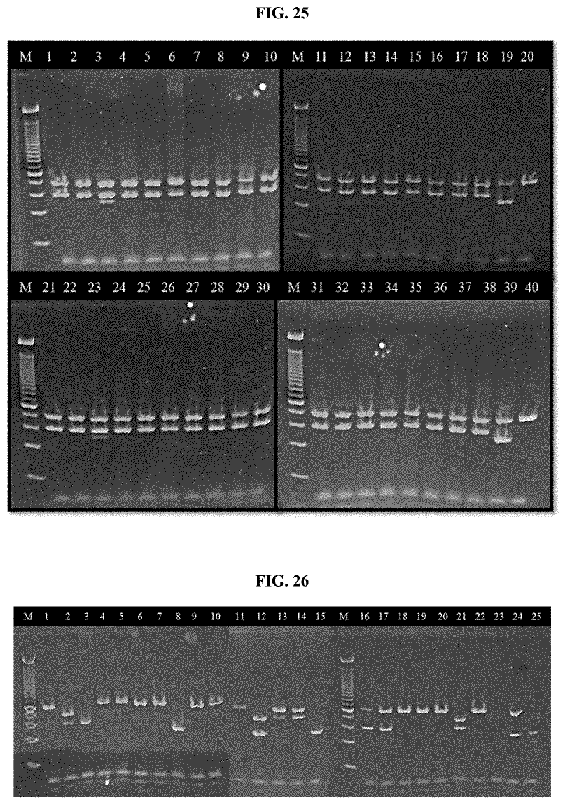

FIG. 25 shows successful PCR amplification from DNA isolated from 19 different clinical samples, shown in Table 2, using this method.

TABLE 2
Samples used in FIG. 25

| Condition | Sample | Dilution factor |
|---|---|---|
| 1 | Positive Control | No dilution |
| 2 | 100 | No dilution |
| 3 | 101 | No dilution |
| 4 | 103 | No dilution |
| 5 | 104 | No dilution |
| 6 | 108 | No dilution |
| 7 | 110 | No dilution |
| 8 | 112 | No dilution |
| 9 | 113 | No dilution |
| 10 | 114 | No dilution |
| 11 | 118 | No dilution |
| 12 | 119 | No dilution |
| 13 | 121 | No dilution |
| 14 | 122 | No dilution |
| 15 | 123 | No dilution |
| 16 | 125 | No dilution |
| 17 | 126 | No dilution |
| 18 | 127 | No dilution |
| 19 | 106 | No dilution |
| 20 | 171 | No dilution |
| 21 | Positive Control | 1:3 |
| 22 | 100 | 1:3 |
| 23 | 101 | 1:3 |
| 24 | 103 | 1:3 |
| 25 | 104 | 1:3 |
| 26 | 108 | 1:3 |
| 27 | 110 | 1:3 |
| 28 | 112 | 1:3 |
| 29 | 113 | 1:3 |
| 30 | 114 | 1:3 |
| 31 | 118 | 1:3 |
| 32 | 119 | 1:3 |
| 33 | 121 | 1:3 |
| 34 | 122 | 1:3 |
| 35 | 123 | 1:3 |
| 36 | 125 | 1:3 |
| 37 | 126 | 1:3 |
| 38 | 127 | 1:3 |
| 39 | 106 | 1:3 |
| 40 | 171 | 1:3 |

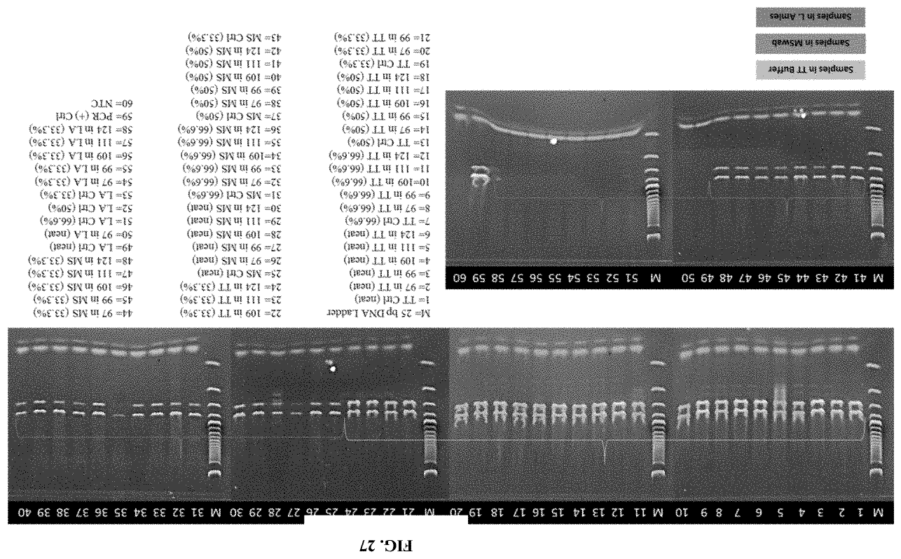

FIG. 26 shows the results of PCR amplification on DNA extracted from the samples in Table 3. Samples in Table 2 were purified in buffer comprising 50 mM Tris pH 8.4, Tween-80, 2% (w/v), BSA, 0.25% (w/v), Proclin 300 0.03% (w/v), and Antifoam SE-15, 0.002% (v/v) made up in purified water, (TT buffer). Amplification was seen in every sample indicating that the PCR reaction possesses high tolerance to inhibitors.

TABLE 3
Samples used in FIG. 26

| | | Microorganism |
|---|---|---|
| Condition | Sample | present |
| 1 | Control NS cells | NS |
| 2 | 97 | |
| 3 | 170 | NG |
| 4 | 172 | CT |
| 5 | 174 | NS |
| 6 | 175 | NS |
| 7 | 176 | NS |
| 8 | 177 | TV |
| 9 | 178 | NS |
| 10 | 179 | NS |
| 11 | 180 | NS |
| 12 | 271 | CT |
| 13 | 272 | CT |
| 14 | 273 | CT |
| 15 | 285 | NG |
| 16 | 288 | NG |
| 17 | 289 | NG |
| 18 | 340 | NS |
| 19 | 341 | NS |
| 20 | 342 | NS |
| 21 | 109 | |
| 22 | Control NS cells | NS |
| 23 | Control NS cells | NS |
| 24 | PCR positive | |
| | control | |
| 25 | No template | |
| | control | |

FIG. 27 depicts the result of an experiment comparing different sample buffers. The sample buffers used were the TT buffer described above, MSwab buffer (MS; Copan Diagnostics, CA), and Liquid Amies Buffer (LA; Copan Diagnostics, CA). PCR products were run on 4% agarose gels to determine the success of the PCR reaction. Samples rehydrated in TT buffer amplified as expected, equal to the controls. The other two medias MS and LA showed varying results, suggesting variable inhibition of the PCR by contaminants from the sample buffer.

### Example 3 Affinity Bead Pull Down of Virus Particles

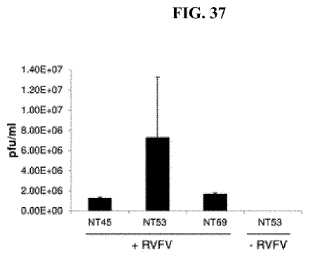

Affinity nanoparticles were prepared with seven different affinity baits. The affinity nanoparticles were incubated with viral supernatants containing Rift Valley fever virus (RVFV, 1E+7 pfu/ml) for 30 minutes at room temperature and washed 4 times with water. Viral RNA was extracted from the particles with Ambion's MagMax Viral RNA extraction kit and quantitated by qRT-PCR assays. All seven affinity baits pulled down viral nucleic acid as shown by the results in FIG. 36. To determine whether the particles were pulling down intact viral particles rather than naked nucleic acid from lysed viral particles a plaque forming assay was conducted. Viral supernatants were incubated with NT46, NT53, and NT69 for 30 minutes at room temperature and washed 4 times with water. Captured viruses were not eluted off of the NanoTrap particles, but rather the samples were diluted and added directly to Vero cells (a kidney epithelial cell line) during the plaque assay procedure. All three affinity nanoparticles tested were capable of pulling down intact infectious viral particles and causing plaques as compared to a control sample without viral particles (−RVFV), as shown in FIG. 37. Further details about viral pull down with affinity particles, such as those in this example, may be found in Shafagati N, et al. (2013) The Use of NanoTrap Particles as a Sample Enrichment Method to Enhance the Detection of Rift Valley Fever Virus. PLOS Neglected Tropical Diseases 7(7): e2296.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The devices and methods described herein are not limited to performing a molecular diagnostic test on human samples. In some embodiments, any of the devices and methods described herein can be used with veterinary samples, food samples, and/or environmental samples. Although the fluid transfer assemblies are shown and described herein as including a piston pump (or syringe), in other embodiments, any suitable pump can be used. For example, in some embodiments any of the fluid transfer assemblies described herein can include any suitable positive-displacement fluid transfer device, such as a gear pump, a vane pump, and/or the like.

1.-21. (canceled)

22. An apparatus, comprising:
a housing;
a reverse transcription module disposed within the housing and configured to receive a sample, the reverse transcription module including a first flow member and a first heater, the first flow member defining an reverse transcription flow path having an inlet portion configured to receive the sample, the first heater fixedly coupled to the first flow member such that the first heater and the reverse transcription flow path intersect at multiple locations, the reverse transcription module configured to perform a reverse transcriptase reaction on the sample;
an amplification module disposed within the housing and configured to receive an output from the reverse transcription module, the amplification module including a second flow member and a second heater, the second flow member defining an amplification volume, the second heater coupled to the second flow member, the amplification module configured amplify the output from the reverse transcription module to produce a target amplicon; and
a detection module disposed within the housing and configured to receive an output from the amplification module, the detection module configured to react a reagent with the target amplicon to produce a signal indicating a presence of the target amplicon.

23. The apparatus of claim 22, further comprising:
a reagent stored within the housing, the reagent formulated to produce the signal that indicates the presence of the target amplicon in the sample, the signal being a colorimetric signal.

24. The apparatus of claim 23, further comprising:
a reagent actuator configured to convey the the reagent from a sealed reagent container into a holding chamber fluidically coupled to the detection module when the reagent actuator is moved from a first position to a second position, the reagent actuator including a locking shoulder configured to matingly engage a portion of the housing to maintain the reagent actuator in the second position.

25. (canceled)

26. The apparatus of claim 22, wherein the detection module is fixedly coupled within the housing and includes a detection surface from which the signal that indicates presence of the target amplicon in the input sample is produced, the detection surface visible via a detection opening defined by the housing.

27.-28. (canceled)

29. The apparatus of claim 22, further comprising:
a power source disposed within the housing and configured to supply power to the amplification module, the power source having a capacity of less than about 1200 mAh.

30.-46. (canceled)

47. The molecular diagnostic test device of claim 22, wherein the reverse transcription module contains a reverse transcriptase enzyme and reagents required for the reverse transcriptase reaction, the reverse transcriptase enzyme and reagents being present as a lyophilized pellet.

48.-51. (canceled)

52. A method for DNA preparation, comprising:
(a) obtaining a biological sample comprising one or more biological entities, wherein the biological entities comprise RNA;
(b) lysing said one or more biological entities, thereby releasing a plurality of RNA molecules therefrom; and
(c) performing a reverse transcriptase reaction on the released RNA molecules to produce a plurality of DNA molecules, wherein said method extracts said nucleic acid molecules from said one or more biological entities within 5 minutes or less at a quality sufficient to successfully perform a polymerase chain reaction (PCR).

53. The method of claim 52, wherein the method is performed by a handheld device.

54. The method of claim 52, wherein a quality sufficient to successfully perform a polymerase chain reaction comprises nucleic acid molecules which amplify with at least 70% efficiency as determined by a qPCR standard curve.

55. The method of claim 52, wherein the method produces at least 100 μI, of a solution containing the nucleic acid molecules.

56.-64. (canceled)

65. A method of DNA preparation, comprising:
conveying a biological sample comprising RNA into a sample input module of a molecular diagnostic test device; and
actuating the molecular diagnostic test device to:
convey the biological sample to a reverse transcription module within the molecular diagnostic test device, the reverse transcription module including a heater and defining a first reaction volume and a second reaction volume, and further comprising lyophilized reagents for a reverse transcription reaction;
maintain an input solution containing the biological sample and the reagents for reverse transcription within the first reaction volume to reverse transcribe at least a portion of the biological sample thereby producing a plurality of complementary DNA (cDNA) molecules;
activate the heater to heat a portion of the reverse transcription module to produce an inactivation temperature zone within the second reaction volume; and
produce a flow of the input solution within the second reaction volume such that a volume of the input solution is heated within the inactivation temperature zone to inactivate an enzyme within the input solution.

66. The method of claim 65, wherein the volume of the input solution is at least 10 microliters.

67. The method of claim 66, wherein the volume of the input solution is produced within five minutes or less.

68. The method of claim 65, wherein the second reaction volume is a serpentine flow path.

69. The method of claim 65, wherein a wall of the reverse transcription module that defines the second reaction volume has a surface area, a ratio of the surface area to the second reaction volume being greater than about 10 cm-1.

70. The method of claim 65, wherein the volume of the input solution is heated to an inactivation temperature of between about 57 degrees Celsius and about 100 degrees Celsius for a time period of at least about 15 seconds.

71.-73. (canceled)

74. The method of claim 65, wherein the portion of the reverse transcription module is a second portion, the actuating the molecular diagnostic test device further causes the molecular diagnostic test device to:
heat a first portion of the reverse transcription module to produce a lysing temperature zone within the second reaction volume, the flow of the input solution within the second reaction volume being such that the volume of the input solution is heated within the lysing temperature zone to lyse a biological entity within the volume of the input solution.

75.-78. (canceled)

79. The method of claim 65, wherein the actuating the molecular diagnostic test device further causes the molecular diagnostic test device to:
heat a portion of an amplification module within the molecular diagnostic test device to amplify a nucleic acid from the plurality of cDNA molecules to produce an output containing a target amplicon; and
convey the output to a detection module of the molecular diagnostic test device.

80. The method of claim 79, further comprising:
reading from the molecular diagnostic test device a signal indicating a presence of the target amplicon; and
discarding, after the reading, the molecular diagnostic test device.

81. An apparatus, comprising:
a housing;
a sample input module defining an input reservoir configured to receive a biological sample, the biological sample containing a biological entity;
a reverse transcription module disposed within the housing, the reverse transcription module including a heater and first flow member, the first flow member defining a first volume and a second volume, the first volume configured to receive an input solution containing at least the biological sample and a lysis buffer, the first volume further containing a lyophilized reverse transcription reagent, the heater coupled to the first flow member and configured to convey thermal energy into the second volume to A) reverse transcribe at least a portion a plurality of ribonucleic acid (RNA) molecules released from the biological sample to produce to convert the plurality of RNA molecules into a plurality of complementary DNA (cDNA) molecules and B) inactivate an enzyme within the input solution or within the lyophilized reverse transcription reagent when a volume of the input solution flows through the second volume; and
an amplification module disposed within the housing, the amplification module including a second flow member configured to receive the volume of the input solution from the reverse transcription module, the amplification module configured to amplify a nucleic acid molecule from the plurality of cDNA molecules within the volume of the input solution to produce an output containing a target amplicon.

82. The apparatus of claim 81, wherein the second volume of the reverse transcription module is a serpentine flow path.

83. The apparatus of claim 81, wherein a wall of the reverse transcription module that defines the second volume has a surface area, a ratio of the surface area to the second volume being greater than about 10 cm-1.

84. The apparatus of claim 81, wherein:
the first volume is in fluid communication with the second volume; and
the reverse transcription module defines a vent opening into the first volume.

85. (canceled)

86. The apparatus of claim 81, wherein:
the heater is a first heater;
the second flow member defines an amplification flow path; and
the amplification module includes a second heater different from the first heater, the second heater coupled to the second flow member and configured to convey thermal energy into the amplification flow path to amplify the plurality of cDNA molecules.

87. The apparatus of claim 81, further comprising:
a fluid pump disposed within the housing, the fluid pump configured to produce a flow of the input solution from the reverse transcription module to the amplification module.

88.-94. (canceled)

95. The apparatus of claim 22, wherein the apparatus is a self-contained device configured to operate without any external instrument.

96. The apparatus of claim 24, further comprising:
a controller disposed within the housing, the controller implemented in at least one of a memory or a processor, the controller including a thermal control module configured to produce a thermal control signal to adjust an output of the heater,
a power source being electrically isolated from the processor when the reagent actuator is in the first position, the power source being electrically coupled to at least one of the processor or the amplification module when the reagent actuator is in the second position.

97. The apparatus of claim 22, further comprising:
a fluid pump disposed within the housing, the fluid pump configured to generate, within the housing, a force that causes a flow of the output produced by the amplification module.

98. The apparatus of claim 81, wherein the apparatus is a self-contained device configured to operate without any external instrument.
